(19) United States
(12) Patent Application Publication
(10) Pub. No.: US 2015/0064111 A1
(43) Pub. Date: Mar. 5, 2015

(54) NOVEL TARGETING AGENTS FOR DIAGNOSTIC AND THERAPEUTIC INDICATIONS

(71) Applicant: APOSENSE LTD., Petach-Tikva (IL)

(72) Inventors: Joel M. Van Gelder, Jerusalem (IL); Menashe Levy, Cesaria (IL); Mirit Argov, Kibbutz Einat (IL); Miri Ben-Ami, Hod Hasharon (IL); Ilan Ziv, Kfar Saba (IL)

(73) Assignee: APOSENSE LTD., Petach-Tikva (IL)

(21) Appl. No.: 14/390,031

(22) PCT Filed: Apr. 3, 2013
(86) PCT No.: PCT/IL13/50307

§ 371 Date: Oct. 2, 2014

Related U.S. Application Data
(60) Provisional application No. 61/619,448, filed on Apr. 3, 2012.

(51) Int. Cl. G01N 33/50; C07C 63/16; C07C 57/58; A61K 51/04; C07D 277/58; C07D 491/147; C07C 233/32; C07C 57/52; C07D 233/91

(52) U.S. Cl. CPC G01N 33/5091; C07C 57/52; C07C 63/16; C07C 57/58; C07D 233/91; C07D 277/58; C07D 491/147; C07C 233/32; A61K 51/0453; A61K 51/0402; G01N 2800/7033; G01N 2510/00
USPC 424/1.89; 562/595; 562/480; 562/489; 548/341.5; 548/191; 546/47; 562/457; 435/35

(57) ABSTRACT

The invention relates to compounds and use thereof in the diagnosis and/or in treatment of medical disorders. In some embodiments, the compounds may be used for detecting a cancer. The compound may include a di-acid moiety. In some embodiments the di-acid moiety comprises a di-carboxylic acid and in some embodiments the di-acid moiety comprises a di-tetrazole.

### FIELD OF THE INVENTION

The invention relates to compounds and use thereof in the diagnosis and/or in treatment of medical disorders.

### BACKGROUND OF THE INVENTION

The plasma membrane (outer membrane) of intact eukaryotic cells is characterized by a highly organized structure. This high level of membrane organization is determined, among others, by the molecular structure of the specific lipids constituting the membrane, the ratio between the various lipid species from which the membrane is composed, the distribution of the phospholipids between the outer and inner leaflets of the membrane and by the membrane protein constituents.

While maintenance of the high level of plasma membrane organization is fundamental to normal cell physiology, substantial perturbations and alterations of the normal organization of the cell plasma membrane (PNOM) occur in numerous physiological and pathological conditions and characterize a plurality of diseases. Such alterations and perturbations may be evident both at the morphological level (membrane blebbing observed in cells undergoing apoptosis) and at the molecular level. PNOM includes, among others, scrambling and redistribution of the membrane phospholipids, with movement to the cell surface of aminophospholipids, mainly phosphatidylserine (PS) and phosphatidylethanolamine (PE), which are normally restricted almost entirely to the inner leaflet of the membrane bilayer, and reciprocal movement of sphingomyelin (SM) and phosphatidylcholine (PC) from the outer leaflet to the inner leaflet of the membrane. This redistribution may be referred to as loss of cell membrane lipid asymmetry. In addition to loss of cell membrane lipid asymmetry, PNOM is also often associated with reduction in the level of packing of membrane phospholipids and an increase in membrane fluidity.

These alterations play an important role in rendering the cell surface a catalytic platform for the assembly of several clotting factor complexes, such as the tenase and prothrombinase protein complexes. Accordingly, platelet activation is associated with the platelet membrane undergoing PNOM, and these alterations constitute an important factor in normal blood coagulation, as well as in the initiation and/or propagation of abnormal, excessive blood clotting in numerous disorders. These disorders include, among others, arterial or venous thrombosis or thrombo-embolism [e.g., cerebral stroke, myocardial infarction, deep vein thrombosis (DVT), disseminated intravascular coagulation (DIC), thrombotic thrombocytopenic purpura, etc.], unstable atherosclerotic plaques, sickle cell disease, beta-thalassemia, anti-phospholipid antibody syndrome [among others in systemic lupus erythematosus (SLE)], and disorders associated with shedding of membrane microparticles, e.g., neurological dysfunction in association with cardiopulmonary bypass.

Apoptosis another major situation in which alterations/perturbations of cell membrane take place. Apoptosis an intrinsic program of cell self-destruction or “suicide”, which is inherent in every eukaryotic cell. In response to a triggering stimulus, cells undergo a highly characteristic cascade of events of cell shrinkage, blebbing of cell membranes, chromatin condensation and fragmentation, culminating in cell conversion to clusters of membrane-bound particles (apoptotic bodies), which are thereafter engulfed by macrophages. PNOM is a universal phenomenon of apoptosis. It occurs early in the apoptotic cascade, probably at the point of cell commitment to the death process, and has also been shown to be an important factor in the recognition and removal of apoptotic cells by macrophages.

A strong correlation has been recently drawn between PNOM and the potent procoagulant activity of apoptotic cells. PNOM in apoptotic endothelial cells, such as those occurring in atherosclerotic plaques, probably plays an important role in the pathogenesis of thrombotic vascular disorders.

Apoptotic cells are also found within tumors. Due to accelerated cell proliferation in tumors, not all cells receive adequate blood supply, resulting in ischemia and increased apoptosis within the tumor. Recognizing this phenomenon, existing apoptotic cells within a tumor may be targeted by specific apoptosis makers for the identification of tumors and for following the biological behavior of tumors on the cellular level, providing, for example, early assessment of the biological effect of treatment on tumors.

Since apoptosis and thrombosis each have an important role in the majority of medical disorders, it is desirable to have tools for detection of these biological processes. Apoptosis specific compounds may be used for detecting apoptotic cells and for targeting apoptotic cells for diagnostic and/or therapeutic purposes.

Compounds having malonic acid substructures for selective binding to PNOM-membranes, are described in WO 2005/067388 to Ziv et al. Specifically, compound ML-10, a (5-fluoropentyl)(methyl)propanedioic acid (or 5-fluoropentyl-2-methyl-malonic acid) was used.

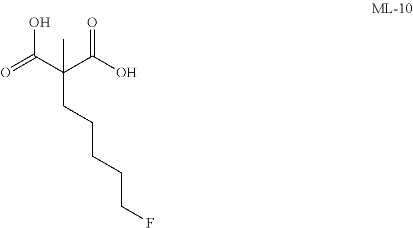

Although tools for targeting tumors and other pathologies are known, improved, more specific and efficient targeting and therapy may be beneficial.

### BRIEF DESCRIPTION OF THE DRAWINGS

Embodiments of the invention will be understood and appreciated more fully from the following detailed description in conjunction with the figures, which are not to scale, in which like reference numerals indicate corresponding, analogous or similar elements, and in which:

FIG. 1 shows the uptake of six exemplary compounds according to the invention in vitro compared to ML-10 in CD95-treated Jurkat cells. Uptake was measured using beta counter. zVAD treated cells reversed the phenotype decreasing uptake to control values (set as 1) in all tested candidates. Shown are means±SD of three independent experiments performed in triplicates.

FIG. 2 shows the tumor/blood uptake ratio of APO-650 in comparison to ML-10. Athymic nude mice were inoculated with HCT-116 human colorectal cancer cells. Mice were subjected to intravenous injection of tritiated ML-10 and APO-650 one and a half hours before samples collection. Blood samples were taken before sacrificing the animals. Blood and tumor samples radioactivity was measured in a beta-counter.

### DETAILED EMBODIMENTS OF THE INVENTION

The present invention provides compounds for selective binding to cells undergoing perturbation of the normal organization of their plasma membrane (PNOM-cells). The compounds of the invention have the advantage of selectively targeting PNOM-cells while also featuring a relatively low molecular weight, and a potentially favorable pharmacokinetic profile.

According to one embodiment of the invention, the compound includes a di-acid moiety, wherein the first logarithmic acid dissociation constant (pKa1) of the di-organic acid moiety is in between about 1.5 and 3.9 and the second logarithmic acid dissociation constant (pKa2) is in between about 5.0 and 6.5. In some embodiments the di-acid moiety comprises a di-carboxylic acid and in some embodiments the di-acid moiety comprises a di-tetrazole.

Compounds according to embodiments of the invention may be used in diagnostics. In some embodiments they may be used for detecting cancer. According to one embodiment of the invention, a +compound may include a marker that may be used with imaging techniques such as X-ray, CT scan, magnetic resonance imaging (MRI) or radio-isotope scan such as single photon emission tomography (SPECT) or positron emission tomography (PET).

In one embodiment, the marker is a detectable label such as the respective radio-isotopes of the metal ions Tc, oxo-Tc, In, Cu, Ga, Xe, Tl and Re, oxo-Re and the covalently linked atoms: ¹²³I and ¹³¹I for radio-isotope scan such as SPECT; Gd(III), Fe(III) or Mn(II) for MRI; and ¹⁸F, ¹⁵O, ¹⁸O, ¹¹C, ¹³C, ¹²⁴I, ¹³N and ⁷⁵Br for PET scan.

In another embodiment, a compound according to embodiments of the invention comprises or is attached to a medicament or a radioisotope which has a therapeutic effect (i.e., is useful for the treatment of disease). A medicament may include a drug for the treatment of cancer, for example without limitation, a cytotoxic agent, such as, a camptothecin, camptothecin analogue, nitrosurea, or another agent with anti-cancer activity such as, lenalidomide. The therapeutic agent may also be a therapeutic radio-isotope, such as Yttrium 90, Iodine 131, Rhenium 188, Holmium 166, Indium 111, Lutetium 177, or any other radioisotopes emitting radiation, which are useful for therapeutic purposes.

The compounds according to embodiments of the invention are designed in correspondence with the structural alterations of the plasma membranes of PNOM-cells, which distinguish these membranes from the membranes of healthy cells.

The compounds of the invention may be used for the detection and diagnosis of a wide variety of medical conditions, characterized by PNOM-cells. Examples of clinical conditions characterized by PNOM-cells are as follows:

Diseases which are characterized by occurrence of excessive apoptosis, such as degenerative disorders, neurodegenerative disorders (e.g., Parkinson's disease, Alzheimer's disease, Huntington chorea), AIDS, ALS, Prion Diseases, myelodysplastic syndromes, ischemic or toxic insults, graft cell loss during transplant rejection; tumors, and especially highly malignant/aggressive tumors, are also often characterized by enhanced apoptosis in addition to the excessive tissue proliferation.

Diseases manifested by excessive blood clotting, wherein PNOM occurs during platelet activation, and/or during activation of or damage to other cellular elements (e.g., endothelial cells). These diseases include, among others, arterial or venous thrombosis, thrombo-embolism, e.g., myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosus.

Inflammatory disorders, and/or diseases associated with immune-mediated etiology or pathogenesis, auto-immune disorders such as antiphospholipid antibody syndrome, systemic lupus erythematosus, connective tissue disorders such as rheumatoid arthritis, scleroderma; thyroiditis; dermatological disorders such as pemphigus or erythema nodosum; autoimmune hematological disorders; autoimmune neurological disorders such as myasthenia gravis; multiple sclerosis; inflammatory bowel disorders such as ulcerative colitis; vasculitis.

Atherosclerotic plaques, and especially plaques that are unstable, vulnerable and prone to rupture, are also characterized by PNOM-cells, such as apoptotic macrophages, apoptotic smooth muscle cells, apoptotic endothelial cells, and activated platelets. Such activated platelets are encountered in the thrombi, often associated with the unstable atherosclerotic plaque.

The detection may also be carried out in a person already known to have the respective disease, for the purpose of evaluating disease severity and in order to monitor disease course and/or response to various therapeutic modalities. A non-limited example for such monitoring is evaluation of response to anticancer therapy. Since most anti-tumor treatments, such as chemotherapy or radiotherapy exert their effect by induction of apoptosis, detection by compounds of the invention of therapy-induced apoptosis of tumor cells may teach on the extent of sensitivity of the tumor to the anti-tumor agent. This may substantially shorten the lag period between the time of administration of the anti-cancer treatment and the time of proper assessment of its efficacy.

Moreover, the detection may be also used to monitor adverse effects of anti-cancer treatments. A large part of such adverse effects is due to untoward treatment-induced apoptosis in normal, yet sensitive cells, such as those of the gastrointestinal epithelium or the bone marrow hematopoietic system.

In addition, the detection may aim at characterization of intrinsic apoptotic load within a tumor, often correlated with the level of tumor aggressiveness; and may also assist in the detection of metastases, via detection of the intrinsic apoptosis frequently occurring within metastases.

Similarly, compounds of the invention may be useful in monitoring graft survival after organ transplantation, since apoptosis plays a major role in cell loss during graft rejection.

In addition, the detection may aim at monitoring response to cyto-protective treatments, and thus aid in screening and development of drugs which are capable of inhibiting cell loss in various diseases (for example those recited above) by enabling a measure of assessment of cell death.

In accordance with this approach, the present invention is related to a method of detection of PNOM-cells in a cell population, selected from whole body, organ, tissue, tissue culture or any other cell population, the method comprising: (i). contacting the cell population with a compound according to any of the embodiments of the invention; and (ii). determining the amount of compound bound to the cell population, wherein detection of a significant amount of compound bound to a cell within the population indicates that the cell is a PNOM-cell.

In another embodiment of the invention, the present invention is related to a method for the detection of PNOM-cells in a tissue or cell culture sample in vitro or ex-vivo, the method comprising: (i). contacting the sample with a compound according to embodiments of the invention, under conditions enabling binding of the compound to the membranes of PNOM-cells; and (ii). detecting the amount of compound bound to the cells; the presence of a significant amount of bound compound indicating the presence of PNOM-cells within the tissue or cell culture.

The step of detection in the in vitro or ex-vivo studies may be, for example, in the case of fluorescent-labeled compound of the invention, without limitation by using flow cytometric analysis, which permits cell visualization on equipment that is widely commercially available. In an example using fluorescence to visualize cells, a single 15 mW argon ion laser beam (488 nm) is used to excite the FITC fluorescence, and fluorescence data is collected using 530 nm band pass filter to provide a histogram. The percent of fluorescent cells can be calculated, for example using Lysis II software or any other software. The method for detection may be used in an embodiment of the invention for screening therapeutic drugs such as anticancer drugs.

The action of the binding depends inter-alia on the method of measuring the difference in binding. The method of the present invention may be used for the diagnosis of a disease characterized by the occurrence of PNOM-cells, for example, without being limited to any of the diseases mentioned above.

According to one embodiment of the invention, there is provided a compound presented by a general the formula A-B, wherein A is a di-acid head moiety and B is a tail moiety.

A (di-acid head moiety)-B (tail moiety)

wherein the di-acid moiety has a first logarithmic acid dissociation constant (pKa1) of between about 1.5 and 3.9 and a second logarithmic acid dissociation constant (pKa2) of between about 5.0 and 6.5, and wherein the di-acid moiety comprises a di-carboxylic acid or a di-tetrazole selected from the following structures:

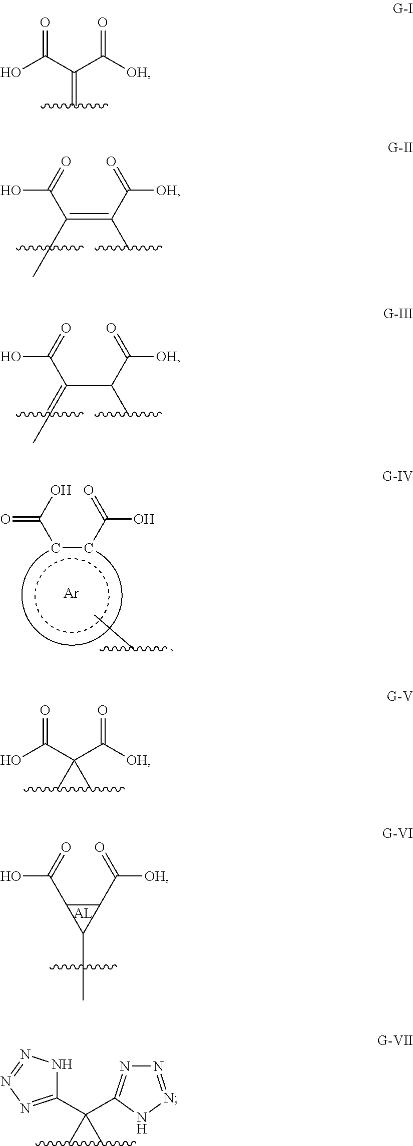

and

wherein the tail moiety may be linear or branched C₁, C₂, C₃, C₄, C₅, C₆, C₇, C₈, or C₉-alkyl or alkylene group optionally substituted with an aryl or heteroaryl comprising one or two rings, wherein said group is possibly attached to a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate, such, as mesylate, tosylate, nosylate or brosylate, or a phenyl substituted by a nitro or halogen. The tail moiety is optionally attached to a marker for diagnostics or therapeutic agent wherein the tail moiety.

A compound according to one embodiment of the invention is represented by formula (I):

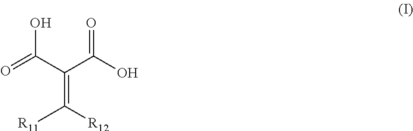

wherein R₁₁ is H or a C₁, C₂ or C₃-alkyl and R₁₂ is a linear or branched C₁, C₂, C₃, C₄, C₅, C₆, C₇, C₈, or C₉-alkyl or alkylene group, wherein each of R₁₁ or R₁₂ is possibly independently attached to a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate, such, as mesylate, tosylate, nosylate or brosylate, or a phenyl substituted by a nitro or halogen. In some embodiments, each of R₁₁ or R₁₂ is possibly independently attached to a marker for diagnostics or therapeutic agent as described above.

According to one embodiment there is provided a compound represented by formula I in which R₁₁ is H and R₁₂ is a C₅-alkyl possibly attached to a marker for diagnostics.

In some embodiments of the invention, the leaving group is a sulfonate such as mesylate, tosylate, nosylate or brosylate, or a phenyl substituted by a nitro or halogen.

According to one embodiment there is provided a compound represented by formula I which is a radiolabeled 2-(5-¹⁸fluoropentylidene) malonic acid.

A compound according to another embodiment of the invention is represented by formula (II):

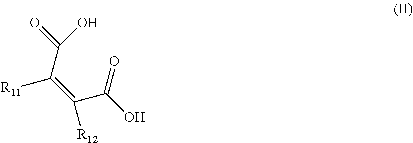

wherein R₁₁ is H or a C₁, C₂ or C₃-alkyl and R₁₂ is a linear or branched C₁, C₂, C₃, C₄, C₅, C₆, C₇, C₈, or C₉-alkyl or alkylene group, wherein each of R₁₁ or R₁₂ is, possibly independently attached to a leaving group, a C₁, C₂, C₃, C₄, C₅ or C₆-alkyl leaving group, or a C₁, C₂, C₃, C₄, C₅ or C₆-alkoxy leaving group; or each of R₁₁ or R₁₂ is possibly independently attached to a marker for diagnostics or therapeutic agent as described above.

According to one embodiment of the invention, there is provided a compound represented by formula II in which R₁₁ is H or CH₃ and R₁₂ is a C₅-alkyl attached to a marker for diagnostics.

According to one embodiment of the invention, there is provided a compound represented by formula II which is a radiolabeled 2-(5-¹⁸fluoropentyl)-3-methylmaleic acid.

A compound according to some embodiments of the invention is represented by formula (III):

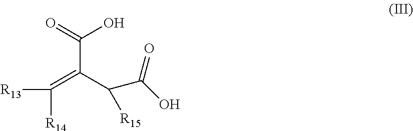

wherein R₁₃ and R₁₄ are independently H, C₁, C₂ or C₃-alkyl and R₁₅ is a linear or branched C₁, C₂, C₃, C₄, C₅, C₆, C₇, C₈, or C₉-alkyl or alkylene group, wherein each of R₁₃, R₁₄ or R₁₅ is, possibly attached to a leaving group, a C₁, C₂, C₃, C₄, C₅ or C₆-alkyl leaving group, or a C₁, C₂, C₃, C₄, C₅ or C₆-alkoxy leaving group, or each of R₁₃, R₁₄ or R₁₅ is possibly attached to a marker for diagnostics or therapeutic agent as described above.

According to one embodiment of the invention, there is provided a compound represented by formula III in which R₁₃ and R₁₄ are CH₃ and R₁₅ is an alkyl attached to a marker for diagnostics.

According to one embodiment there is provided a compound represented by formula III which is a radiolabeled 2-(5-¹⁸fluoropentyl)-3-methylenesuccinic acid.

A compound according to another embodiment of the invention is represented by formula (IV):

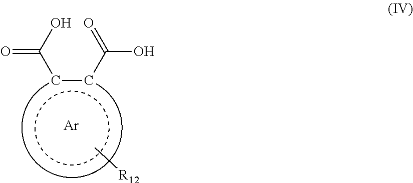

wherein the aromatic ring Ar contains 4, 5 or 6 atoms, in which the aromatic ring atoms that are not substituted by the carboxy groups are independently selected from carbon, nitrogen, oxygen or sulfur and wherein R₁₂ is a fluorine atom, a leaving group or a marker for diagnostics or therapeutic, or R₁₂ is a linear or branched C₁, C₂, C₃, C₄, C₅, C₆, C₇, C₈, or C₉-alkyl or alkylene group, wherein R₁₂ is possibly attached to a leaving group, a C₁, C₂, C₃, C₄, C₅ or C₆-alkyl leaving group, or a C₁, C₂, C₃, C₄, C₅ or C₆-alkoxy leaving group; or wherein R₁₂ is possibly attached to a marker for diagnostics or therapeutic agent as described above.

According to an embodiment of the invention, there is provided a compound represented by formula IV in which Ar is a C₆ ring and R₁₂ is a C₃ or C₅-alkyl attached to a marker for diagnostics.

According to one embodiment of the invention, there is provided compound represented by the formula IVp:

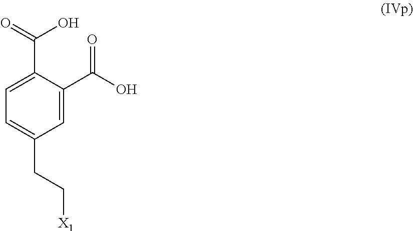

wherein X₁ is selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen. According to an embodiment of the invention, this is a precursor for the radiolabelled molecule.

According to an embodiment of the invention, there is provided a compound represented by formula IVa which is a radiolabeled 4-(5-¹⁸fluoropentyl)phthalic acid.

According to one embodiment of the invention, there is provided a compound represented by formula IVb, also designated as APO-630 (UB-12497) 4-(3-¹⁸fluoropropyl)phthalic acid.

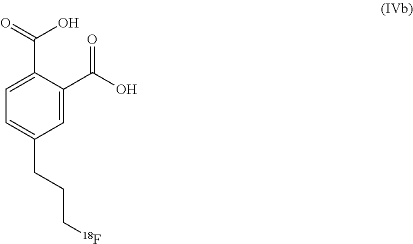 [SMILES: O=C(O)c1ccc(CCC[18F])cc1C(=O)O]

A compound according to another embodiment of the invention is represented by formula (V):

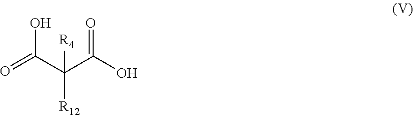

wherein R₄ is H or a C₁, C₂ or C₃-alkyl and R₁₂ is a linear or branched C₁, C₂, C₃, C₄, C₅, C₆, C₇, C₈, or C₉-alkyl or alkylene group, optionally substituted with an aryl or heteroaryl comprising one or two rings, each of R₄ or R₁₂ is possibly attached to a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen; or to a marker or therapeutic agent as described above.

According to one embodiment there is provided a compound represented by formula V in which R₄ is CH₃ and R₁₂ is an alkyl attached to a marker for diagnostics.

According to one embodiment of the invention, there is provided a compound represented by formula V in which R₄ is C₁, C₂ or C₃-alkyl possibly attached to a marker for diagnostics and R₁₂ is a linear or branched C₁, C₂, C₃, C₄, C₅, C₆, C₇, C₈, or C₉-alkyl or alkylene group, optionally substituted with an aryl or heteroaryl comprising one or two rings.

According to one embodiment of the invention, there is provided a compound represented by formula V in which R₄ is CH₃ and R₁₂ is a C₁, C₂, C₃, C₄, C₅, C₆-alkyl attached to a DNA intercalator. In an embodiment of the invention, when the DNA intercalator is a berberine, the compound is designated as APO-681.

According to one embodiment of the invention, there is provided a compound represented by formula V in which R₄ is CH₃ and R₁₂ is a C₁, C₂, C₃, C₄, C₅, C₆-alkyl attached to a tubulin ligand. When in one embodiment, the tubulin ligand is a colchicine, the compound is designated as APO-697.

According to one embodiment of the invention, there is provided a compound represented by formula V in which R₄ is CH₃ and R₁₂ is a C₁, C₂, C₃, C₄, C₅, C₆-alkyl attached to a heat shock protein ligand, such as for example, Hsp90.

According to one embodiment of the invention, there is provided a compound represented by formula V in which R₄ is CH₃ and R₁₂ is a C₁, C₂, C₃, C₄, C₅, C₆-alkyl attached to a DNA methyl transferase inhibitor, such as without being limited, azacytidine.

According to one embodiment of the invention, there is provided a compound which is a radiolabeled 2-(4-(¹⁸fluoromethyl)phenethyl)-2-methylmalonic acid.

According to one embodiment of the invention, there is provided a compound represented by formula Vb also designated as APO-623 (UB-12751). In some embodiments, the APO-623 may be ¹⁸F radiolabeled by a ¹⁸F or ¹⁸F—C₁, C₂, C₃, C₄, C₅, or C₆-alkyl.

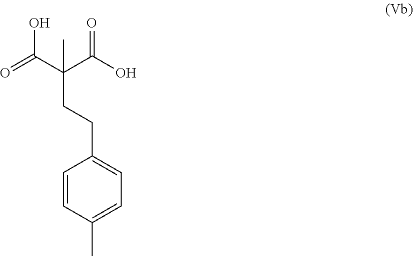 [SMILES: Cc1ccc(CCC(C)(C(=O)O)C(=O)O)cc1]

According to one embodiment of the invention, there is provided a compound represented by the formula Vbp:

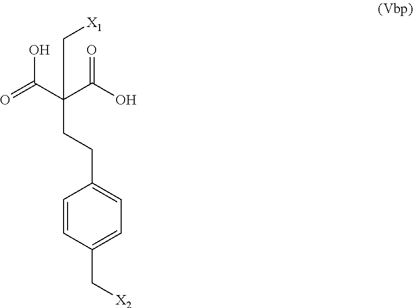

wherein each of X₁ and X₂ is independently selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen. According to one embodiment, the leaving group may be substituted by a ¹⁸F or ¹⁸F—C₁, C₂, C₃, C₄, C₅, or C₆-alkyl.

According to one embodiment of the invention there is provided a compound represented by formula Vb′ in which the APO-623 molecule is ¹⁸F radiolabeled.

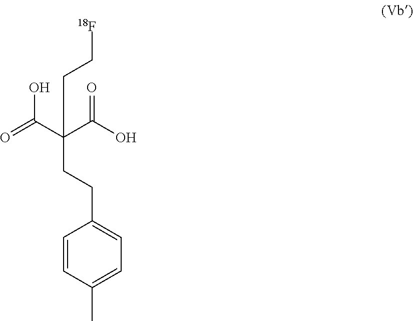 [SMILES: Cc1ccc(CCC(CC[18F])(C(=O)O)C(=O)O)cc1]

According to one embodiment of the invention, there is provided a compound represented by formula Vb″ in which the APO-623 molecule is ¹⁸F radiolabeled.

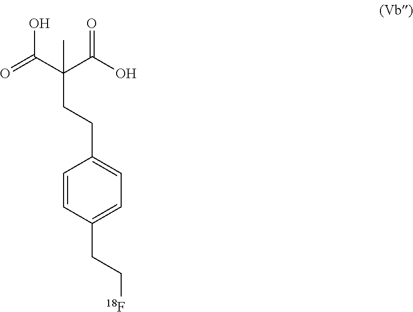 [SMILES: CC(CCc1ccc(CC[18F])cc1)(C(=O)O)C(=O)O]

According to one embodiment of the invention, there is provided a compound represented by formula Vc also designated as APO-646 (UB-12818). In some embodiments, the APO-646 may be ¹⁸F radiolabeled by a ¹⁸F or ¹⁸F—C₁, C₂, C₃, C₄, C₅, or C₆-alkyl.

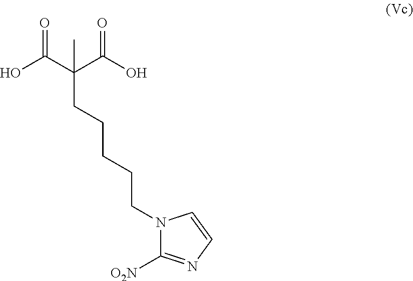 [SMILES: CC(CCCCCn1ccnc1[N+](=O)[O-])(C(=O)O)C(=O)O]

According to one embodiment of the invention, there is provided a compound represented by the formula Vcp:

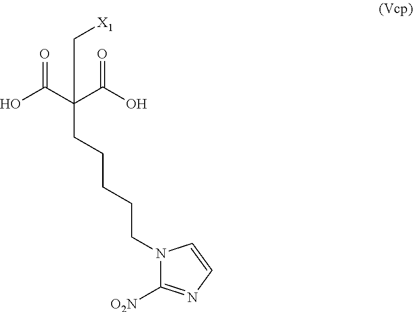

wherein X₁ is selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen.

According to one embodiment of the invention, there is provided a compound represented by formula Vc′ in which the APO-646 molecule is ¹⁸F radiolabeled.

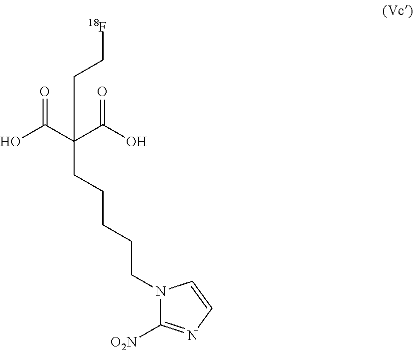 [SMILES: O=C(O)C(CC[18F])(CCCCCn1ccnc1[N+](=O)[O-])C(=O)O]

According to one embodiment of the invention, there is provided a compound represented by formula Vd also designated as APO-650 (UB-13295). In some embodiments, the APO-650 may be ¹⁸F radiolabeled by a ¹⁸F or ¹⁸F—C₁, C₂, C₃, C₄, C₅, or C₆-alkyl.

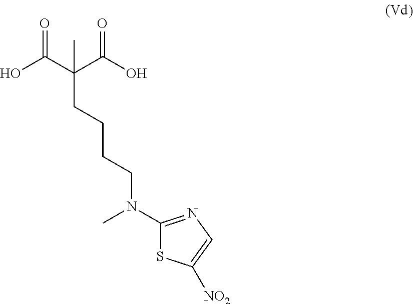 [SMILES: CN(CCCCC(C)(C(=O)O)C(=O)O)c1ncc([N+](=O)[O-])s1]

According to one embodiment of the invention, there is provided a compound represented by the formula Vdp:

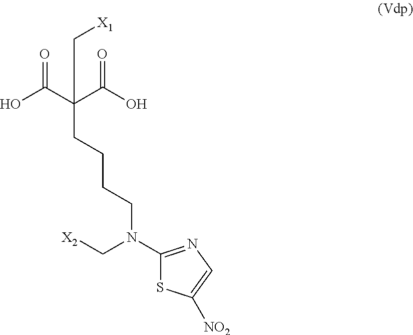

wherein each of X₁ and X₂ is independently selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen.

According to one embodiment of the invention, there is provided a compound represented by formula Vd′ in which the APO-650 molecule is further ¹⁸F radiolabeled.

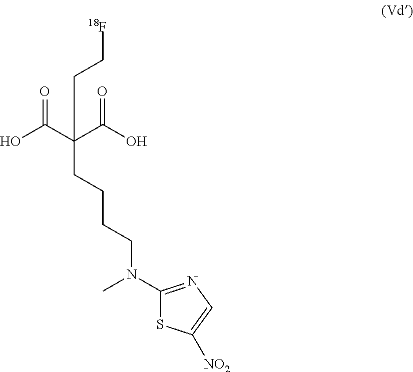 [SMILES: CN(CCCCC(CC[18F])(C(=O)O)C(=O)O)c1ncc([N+](=O)[O-])s1]

According to one embodiment of the invention, there is provided a compound represented by formula Vd″ in which the APO-650 molecule is ¹⁸F radiolabeled.

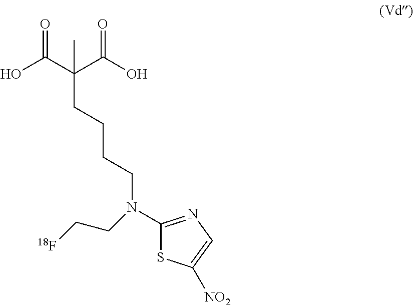 [SMILES: CC(CCCCN(CC[18F])c1ncc([N+](=O)[O-])s1)(C(=O)O)C(=O)O]

According to one embodiment of the invention, there is provided a compound represented by formula Ve also designated as APO-681. In some embodiments, the APO-681 may be ¹⁸F radiolabeled by a ¹⁸F or ¹⁸F—C₁, C₂, C₃, C₄, C₅, or C₆-alkyl.

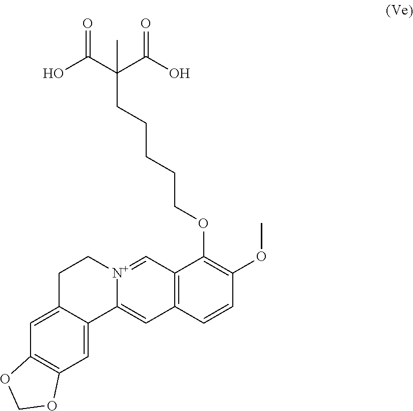 [SMILES: COc1ccc2cc3[n+](cc2c1OCCCCCC(C)(C(=O)O)C(=O)O)CCc1cc2c(cc1-3)OCO2]

According to one embodiment of the invention, there is provided a compound represented by the formula Vep:

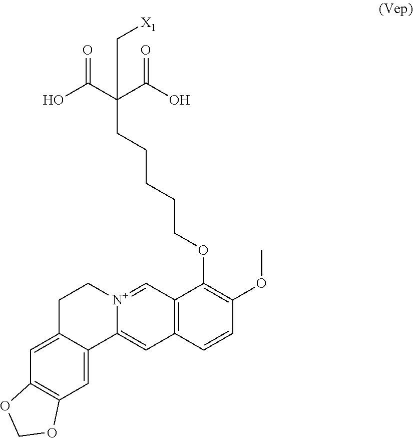

wherein X₁ is selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen.

According to one embodiment of the invention, there is provided a compound represented by formula Ve′ in which the APO-681 molecule is further ¹⁸F radiolabeled.

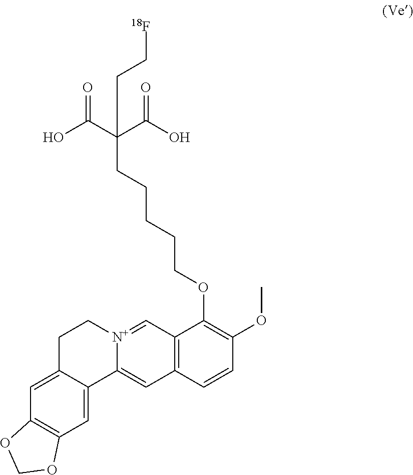 [SMILES: COc1ccc2cc3[n+](cc2c1OCCCCCC(CC[18F])(C(=O)O)C(=O)O)CCc1cc2c(cc1-3)OCO2]

According to one embodiment of the invention, there is provided a compound represented by formula Ve also designated as APO-697. In some embodiments, the APO-697 may be ¹⁸F radiolabeled by a ¹⁸F or ¹⁸F—C₁, C₂, C₃, C₄, C₅, or C₆-alkyl.

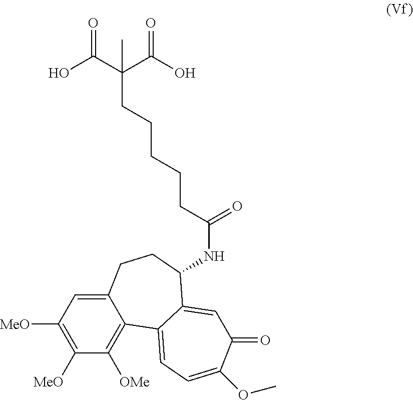 [SMILES: COc1cc2c(c(OC)c1OC)-c1ccc(OC)c(=O)cc1[C@@H](NC(=O)CCCCCC(C)(C(=O)O)C(=O)O)CC2]

According to one embodiment of the invention, there is provided a compound represented by the formula Vfp:

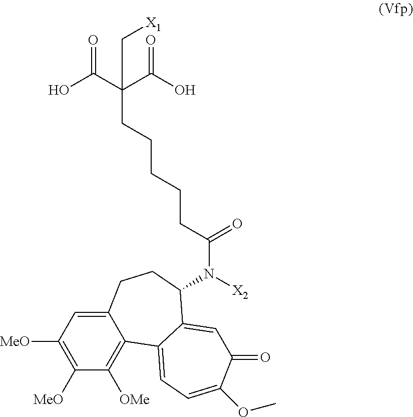

wherein each of X₁ and X₂ is independently selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen.

According to one embodiment of the invention, there is provided a compound represented by formula Vf′ in which the APO-697 molecule is ¹⁸F radiolabeled.

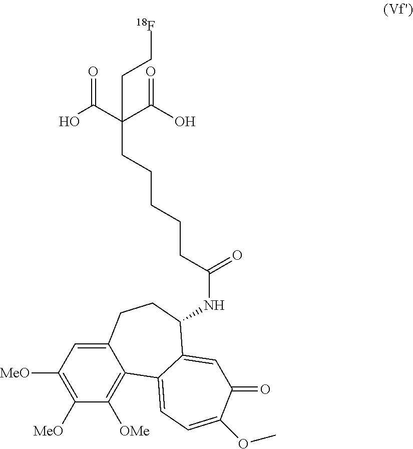 [SMILES: COc1cc2c(c(OC)c1OC)-c1ccc(OC)c(=O)cc1[C@@H](NC(=O)CCCCCC(CC[18F])(C(=O)O)C(=O)O)CC2]

According to one embodiment of the invention, there is provided a compound represented by formula Vf″ in which the APO-697 molecule if further ¹⁸F radiolabeled.

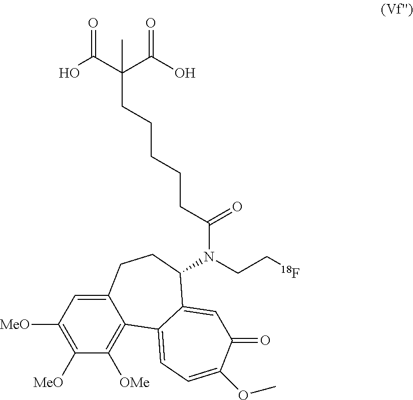 [SMILES: COc1cc2c(c(OC)c1OC)-c1ccc(OC)c(=O)cc1[C@@H](N(CC[18F])C(=O)CCCCCC(C)(C(=O)O)C(=O)O)CC2]

A compound according to another embodiment of the invention is represented by formula (VI):

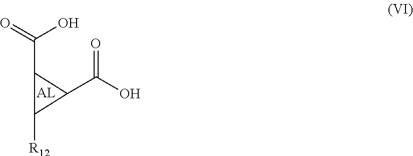

wherein the cycloalkyl AL contains 3, 4, 5 or 6 carbons and R₁₂ is a linear or branched C₁, C₂, C₃, C₄, C₅, C₆, C₇, C₈, or C₉-alkyl or alkylene group, possibly attached to a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen; or R₁₂ is possibly attached to a marker for diagnostics or therapeutic agent as described above.

A compound according to another embodiment of the invention is represented by formula (VII):

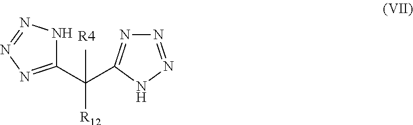

wherein R₄ is H or a C₁, C₂ or C₃-alkyl and R₁₂ is a linear or branched C₁, C₂, C₃, C₄, C₅, C₆, C₇, C₈, or C₉-alkyl or alkylene group, optionally substituted with an aryl or heteroaryl comprising one or two rings, wherein each of R₄ or R₁₂ is possibly attached to a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen; or in some embodiments, each of R₄ or R₁₂ is possibly attached to a marker for diagnostics or therapeutic agent as described above.

According to one embodiment of the invention, R₄ is CH₃ and R₁₂ is a C₄ alkyl attached to a marker for diagnostics.

According to one embodiment of the invention, there is provided a compound represented by formula VIIa which is 5,5′(heptane-2,2-diyl)bis(1H-tetrazole).

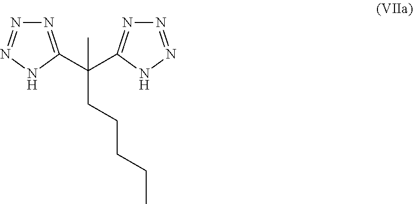 [SMILES: CCCCCC(C)(c1nnn[nH]1)c1nnn[nH]1]

According to one embodiment of the invention, there is provided a compound represented by the formula VIIap:

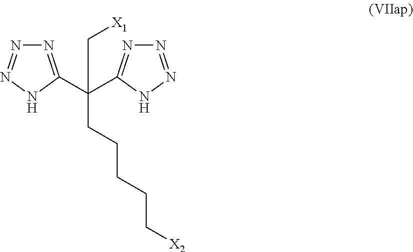

wherein each of X₁ and X₂ is independently selected from H, a leaving group, a C₁, C₂, C₃, C₄, C₅ or C₆-alkyl leaving group, or a C₁, C₂, C₃, C₄, C₅ or C₆-alkoxy leaving group; the leaving group may be a sulfonate such as mesylate, tosylate, nosylate or brosylate, or a phenyl substituted by a nitro or halogen.

According to one embodiment of the invention, there is provided a compound represented by formula VIIa′ in which the molecule is further ¹⁸F radiolabeled, and is designated as 5,5′(7-¹⁸fluoroheptane-2,2-diyl)bis(1H-tetrazole)), also designated as APO-600 (UB-12495).

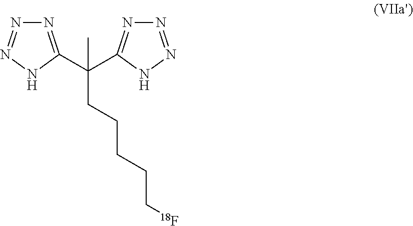 [SMILES: CC(CCCCC[18F])(c1nnn[nH]1)c1nnn[nH]1]

According to some embodiments of the invention, there is provided a method for detecting cancer in a subject comprising administering the compound of the invention, wherein the compound is attached to a marker for diagnostics, to a subject; and imaging the subject using an imaging technique selected from one or more of X-ray, CT scan, magnetic resonance imaging (MRI), single photon emission tomography (SPECT) and positron emission tomography (PET).

According to some embodiments of the invention, there is provided a method for monitoring a treatment for cancer in a subject comprising administering the compound according to the embodiments of the invention, wherein the compound is attached to a marker for diagnostics, to a subject before, during and after a chemotherapeutic treatment and imaging the subject using an imaging technique selected from one or more of X-ray, CT scan, magnetic resonance imaging (MRI), single photon emission tomography (SPECT) and positron emission tomography (PET), wherein a decrease signal of imaging after or during is indicative that the chemotherapeutic treatment is successful.

In some embodiments of the invention, there is provided a kit comprising the compounds of the invention, in the non radiolabeled state together with means for radiolabeling.

Due to the short half-life of certain radio-isotopes used as markers for imaging, such as ¹⁸F, the attachment of such marker for the purposes of, for example, clinical PET imaging may be performed immediately before the administration of the diagnostic compound to the patient. Therefore, it may be useful to synthesize a precursor, comprising a moiety to be substituted by a radio-isotope, such as ¹⁸F, before administration to the patient.

Compound 5 in Example 1, compound 5 in Example 5 and compound 5 in Example 6 are examples of precursors according to embodiments of the invention.

In one embodiment there is provided a precursor compound of formulae (VIII):

R—O—X (VIII)

in which R stands for any of the compounds described above and X is a leaving group such as an halide as Br or Cl or a sulfonate such as mesylate, tosylate and triflate. According to some embodiments functional groups of the compounds may be protected by protecting groups.

Two exemplary precursors are shown in compounds of formulae VIII′ and VIII″.

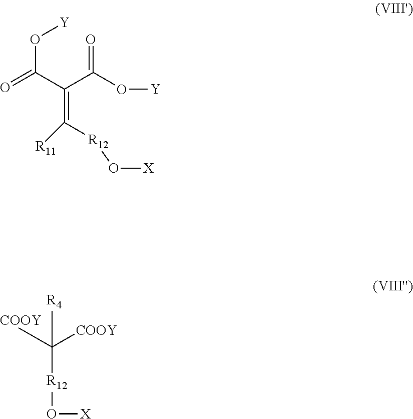

in which R₁₁ and R₄ are each independently H or a C₁, C₂ or C₃ alkyl and R₁₂ is a linear or branched C₁, C₂, C₃, C₄, C₅, C₆, C₇, C₈, or C₉-alkyl or alkylene optionally substituted with an aryl or heteroaryl comprising one or two rings; Y is a protecting group such as methyl, ethyl, tert-butyl, benzyl and X is a leaving group such as an halide as Br or Cl or a sulfonate such as mesylate, tosylate and triflate.

The method for labeling a precursor with ¹⁸F for PET imaging comprises the step of attaching an ¹⁸F atom to the precursor; thereby radio-labeling the compound with ¹⁸F for PET imaging. Attaching an ¹⁸F atom to the precursor may be done by known methods, such as kryptofix-mediated fluorination. Optionally, the functional groups of the precursor may be protected by appropriate protecting groups prior to the step of attaching ¹⁸F atom. Said protecting groups are thereafter optionally removed after the step of attachment of the ¹⁸F atom.

In the case that the marker is a metal atom (e.g., Gd, ^{99m}Tc or oxo-^{99m}Tc for MRI or SPECT, respectively), the compound comprises a metal chelator. The metal coordinating atoms of the chelator may be nitrogen, sulfur or oxygen atoms. In an embodiment of the invention, the chelator is diaminedithiol, monoamine-monoamide-bisthiol (MAMA), triamide-monothiol, and monoamine-diamide-monothiol. In such case, both a compound-chelate precursor, being the compound attached to or comprising a chelator prior to complexation with the metal atom, and the complex comprising the metal atom, are included in the scope of the invention.

For fluorescent detection, a compound of the invention may comprise a fluorescent group selected from any fluorescent probe known in the art. Examples for such probes are 5-(dimethylamino) naphthalene-1-sulfonylamide (dansyl-amide), and fluorescein.

### EXAMPLES

In order to understand the invention and to see how it may be carried-out in practice, the following examples are described: examples directed to synthesis of the compounds of the invention; and examples directed to the performance of the compounds of the invention in selective binding to cells undergoing death process.

### Example 1

### Synthesis of 2-(5-fluoropentylidene) malonic acid (an exemplary embodiment of a compound of formula I)

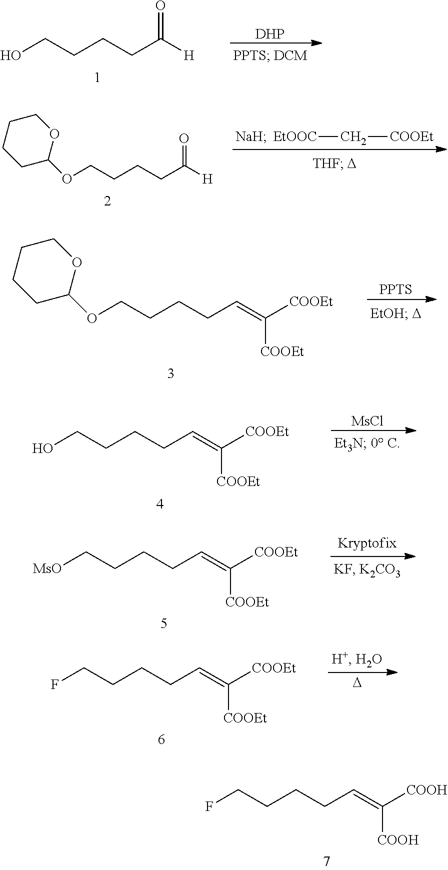

5-Hydroxypentanal (1), 3 g, is treated with 1.5 eq of 3,4-dihydro-2H-pyran and 0.1 eq of pyridinium para toluenesulfonate (PPTS) in 135 mL of CH₂Cl₂. Other hydroxyalkanals (typically, C₂₋₁₀ branched or linear alkyls) may be used as starting compounds in a similar synthesis to obtain other embodiments of compounds of formula I. After work-up and purification, 1.45 g (33%) of product 2 is obtained. 2 eq of NaH are added under nitrogen to a solution of 1.0 eq of diethyl malonate in anhydrous THF and after 10 minutes 1.0 eq of aldehyde 2 is added. The reaction mixture is refluxed and after 10 hours a complete conversion can be observed resulting in a 90% yield. Deprotection of tetrahydropyran (THP) with PPTS is done in ethanol at 55° C. After work-up, a quantitative yield of alcohol 4 can be obtained and directly used for a mesylation reaction. With the mesylate 5 in hand, a kryptofix-mediated fluorination is performed. Compound 6 can be obtained in 75% yield and hydrolyzed under acidic conditions to its corresponding di-acid 7.

¹H NMR [for example, Bruker Avance 400 (400 MHz, CDCl3, TMS as internal standard] of the 2-(5-fluoropentylidene) malonic acid compound shows the following results: δ 1.29 (m, 2H, CH₂), 1.49 (m, 2H, CH₂), 2.18 (m, 2H, CH₂), 4.09 (m, 2H, CH₂F), 7.00 (t, 1H, C═CH), 11.00 (s, 2H, COOH).

### Example 2

### Synthesis of 2-(5-fluoropentyl)-3-methylmaleic acid (an exemplary embodiment of a compound of formula II)

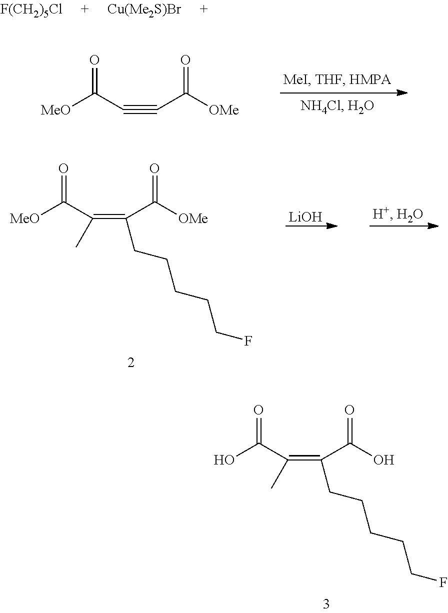

5-Fluoropentyl magnesium chloride (0.6 mL of 1 M solution in THF, 1.2 mmol) is added dropwise to a suspension of cuprous bromide-dimethyl sulfide complex (CuBr.Me₂S, 0.25 g, 1.20 mmol) in THF (6 mL) at −40° C. The resulting suspension is stirred at −40° C. for 2 h and then cooled to −78° C., and freshly distilled dimethyl acetylenedicarboxylate (0.14 g, 1.00 mmol) in THF (2 mL) is added dropwise. The reaction mixture is stirred for 40 min, then HMPA-THF solution (1:1, 2 mL) is added, which results in the heterogeneous mixture becoming nearly homogeneous. Subsequently, MeI (2.5 mmol, 0.36 g, 0.16 mL) in THF (2 mL), is added and stirring is continued for 5 min at −78° C. After warming the mixture to room temperature overnight, saturated aqueous NH₄Cl (2 mL, adjusted to pH 8 with 10% ammonia) is added to the reaction mixture at −20° C. The mixture is stirred at 20° C. for 30 min and then partitioned between ether and water. The aqueous layer is extracted with ether (3×10 mL), and the combined organic extracts are successively washed with additional aqueous NH₄Cl (20 mL), water (2×20 mL) and brine (20 mL). Drying (Na₂SO₄), concentration in vacuo and purification by flash column chromatography may provide 365 mg of compound 2.

Other starting materials, such as B—C₁₋₉ magnesium chloride and cuprous bromide di-(C₁₋₃) sulfide complex, in which B is a marker or other substance such as a medicament, may be used to obtain other embodiments of compounds of formula II.

1.0 N LiOH (2 equiv) is added to compound 2 (50 mg) in THF—H₂O (2 mL, 1:1) and the mixture is stirred at room temperature until the starting material is consumed as indicated by TLC. The solvent is removed in vacuo and the remaining solid is dissolved in acidic H₂O (3 mL). Freeze-drying of this solution gives compound 3 (E and Z isomer).

¹H NMR [for example, Bruker Avance 400 (400 MHz, CDCl₃, TMS as internal standard] of the 2-(5-fluoropentyl)-3-methylmaleic acid compound shows the following results: δ 1.29 (m, 4H, CH₂), 1.49 (m, 2H, CH₂), 2.41 (t, 2H, ═CCH₂), 2.43 (s, 3H, ═CCH₃), 4.09 (m, 2H, CH₂F), 11.00 (s, 2H, COOH).

### Example 3

### Synthesis of 2-(5-fluoropentyl)-3-methylenesuccinic acid (an exemplary embodiment of a compound of formula III)

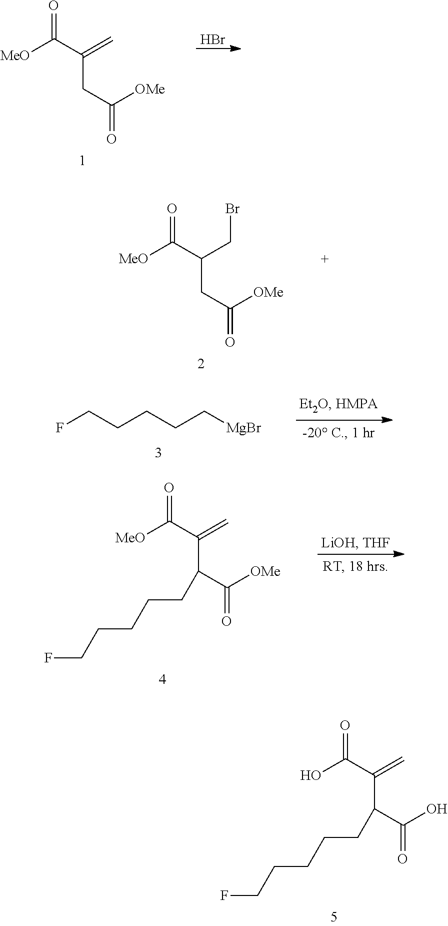

Other starting materials, such as B—C₁₋₉ magnesium bromide complexes, in which B is a marker or other substance such as a medicament, may be used to obtain other embodiments of compounds of formula III.

### Example 4

### Synthesis of 4-(5-fluoropentyl)phthalic acid (an exemplary embodiment of a compound of formula IV)

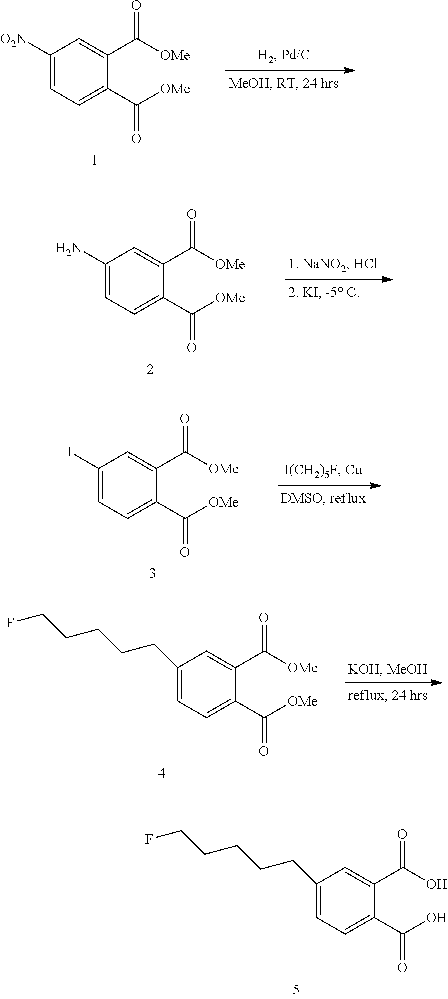 [SMILES: COC(=O)c1ccc([N+](=O)[O-])cc1C(=O)OC ; COC(=O)c1ccc(N)cc1C(=O)OC ; COC(=O)c1ccc(I)cc1C(=O)OC ; COC(=O)c1ccc(CCCCCF)cc1C(=O)OC ; O=C(O)c1ccc(CCCCCF)cc1C(=O)O]

Pd/C (1.02 g) is added to a solution of compound 1 (31.13 g) in MeOH (400 mL) under argon. The flask is then charged with hydrogen gas and the reaction mixture is stirred for 24 hours at room temperature under a hydrogen atmosphere. The reaction mixture is then filtered by using celite, and the filtrate is evaporated under vacuum. Compound 2 can be obtained at a yield of 25.16 g (91%). Dilute HCl (200 mL) is added slowly to the flask containing compound 2 (24.99 g). The reaction mixture is stirred at room temperature for 1 hour, and cooled down to −10° C. A solution of NaNO₂ (11.18 g) in H₂O (70 mL) is then added slowly to the reaction mixture at 5° C. and stirred for 30 min at −10° C. The resulting mixture is added slowly to another flask containing a solution of KI (35.96 g) in H₂O (300 mL). After finishing the addition, the mixture is stirred for 30 min. Then the organic layer is extracted three times by Et₂O, followed by washing with saturated Na₂S₂O₃ solution, and drying over Na₂SO₄. After filtration and evaporation of the solvent, the resulting crude product is purified by silica gel column chromatography with CHCl₃ as an eluent and compound 3 can be obtained at a yield of 29.3 g (80%). A mixture of compound 3 (5.01 g) with 5-fluoropentyl iodide (3.38 g), Cu (2.81 g), 2,2-bipyridine (0.48 g) and DMSO (20 mL), is stirred at 110° C. for 32 hours under argon. After being cooled down the reaction is filtered using celite and the organic substances were extracted with Et₂O, followed by washing with brine and dried over Na₂SO₄. The crude product is purified by silica gel column chromatography using CHCl₃ as the eluent to afford compound 4 at a yield of 7.62 g (83%). A mixture of compound 4 (0.50 g), 30% KOH solution (15 mL), and MeOH (10 mL) is stirred at 90° C. for 24 h. The reaction mixture is then concentrated until the volume is reduced to 10 mL and acidified by concentrated HCl. The resulting mixture is then filtered, washed with CHCl₃, and the solvent is evaporated to afford compound 5 at a yield of 0.455 g (99.5%). Other compounds may be used as starting compounds in a similar synthesis to obtain other embodiments of compounds of formula IV.

¹H NMR [for example, Bruker Avance 400 (400 MHz, CDCl₃, TMS as internal standard] of the 4-(5-fluoropentyl)phthalic acid compound shows the following results: δ 1.29 (m, 2H, CH₂), 1.49 (m, 2H, CH₂), 1.59 (m, 2H, CH₂), 2.62 (m, 2H, CH₂—Ar), 4.09 (m, 2H, CH₂F), 7.37-7.61 (m, 3H, Ar), 11.00 (s, 2H, COOH).

### Example 4a

### Synthesis of 4-(1,1,2,2-tetratritium-3-Fluoropropyl)benzene-1,2-dicarboxylic acid (compound of formula IVa)

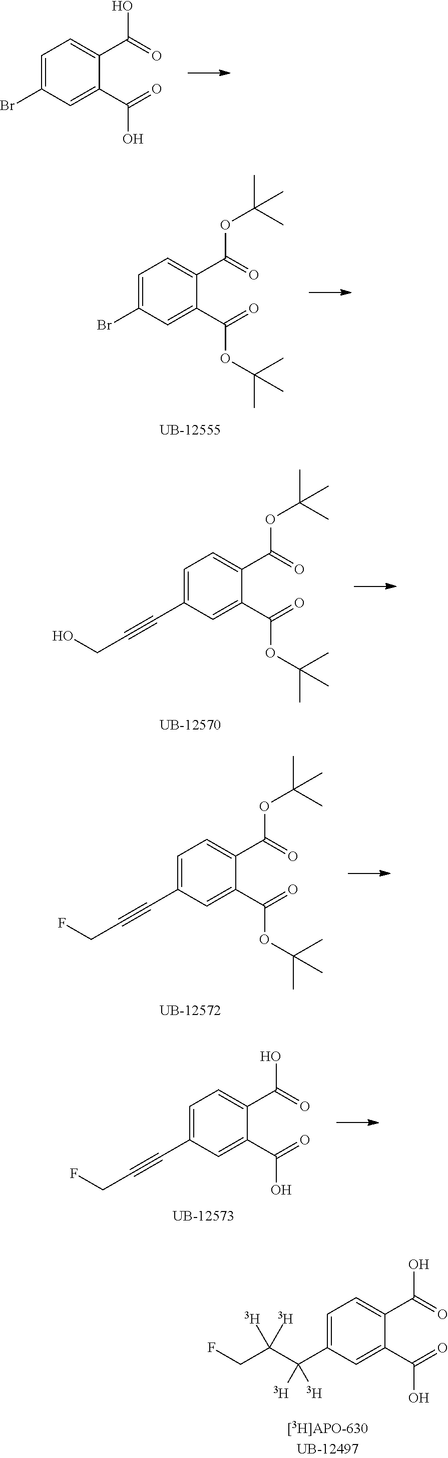 [SMILES: O=C(O)c1ccc(Br)cc1C(=O)O ; CC(C)(C)OC(=O)c1ccc(Br)cc1C(=O)OC(C)(C)C ; CC(C)(C)OC(=O)c1ccc(C#CCO)cc1C(=O)OC(C)(C)C ; CC(C)(C)OC(=O)c1ccc(C#CCF)cc1C(=O)OC(C)(C)C ; O=C(O)c1ccc(C#CCF)cc1C(=O)O ; [3H]C([3H])(CF)C([3H])([3H])c1ccc(C(=O)O)c(C(=O)O)c1]

### Synthesis of 1,2-Di-tert-butyl 4-bromobenzene-1,2-dicarboxylate (UB-12555)

5 g (20.4 mmol, 1 equiv.) of 4-bromophthalic acid and 50 ml of dry DCM were added into a high pressure reactor. Concentrated sulfuric acid (0.5 ml) was added, and the mixture was cooled to −70° C. 30 ml of isobutylene was condensed and added to the mixture in one portion. The reaction mixture was stirred in a closed reactor, at room temperature overnight. The reaction was then assessed by TLC, which showed complete consumption of starting material. The reactor was opened, and the mixture was stirred to release residual isobutylene. To the mixture 60 ml of 10% NaOH solution was added and the DCM was evaporated. The residue was extracted with 2×80 ml of ethyl acetate, the combined organics were washed with brine and dried over Na₂SO₄. After evaporation a yellowish brown oil was obtained. Yield: 7.3 g (20.5 mmol, 100%).

### Synthesis of 1,2-Di-tert-butyl 4-(3-hydroxyprop-1-yn-1-yl)benzene-1,2-dicarboxylate (UB-12570)

7.3 g (20.5 mmol, 1 equiv.) of UB-12555 was dissolved in 75 ml of dry toluene. The solution was degassed and inerted with argon. 1.29 ml (22 mmol, 1.1 equiv) of propargyl alcohol, 0.15 g (0.8 mmol, 0.04 equiv.) of copper(I) iodide, 3.65 ml (26 mmol, 1.3 equiv.) of triethyl amine and 1.16 g (1 mmol, 0.05 equiv.) of tetrakis(triphenyl phosphine)palladium(0) were added to the solution. The reaction mixture was heated to 80° C. and stirred overnight. The mixture was passed through a pad of Celite and the filtrate washed with 2×40 ml of 1M HCl solution. The evaporated crude product was purified by column chromatography (220 g silica gel, hexane:ethyl acetate=3:1). Yield: 3.8 g (11.4 mmol, 55.6%).

### Synthesis of 1,2-Di-tert-butyl 4-(3-fluoroprop-1-yn-1-yl)benzene-1,2-dicarboxylate (UB-12572)

1.3 g (3.9 mmol, 1 equiv.) of UB-12570 was dissolved in 15 ml of DCM, and the solution was cooled to 0° C. 1.2 ml (9 mmol, 2.3 equiv.) of DAST was added to the solution and the cooled reaction mixture was stirred for 1 hour, then allowed to warm to room temperature and stirred overnight. The reaction was complete. The mixture was added dropwise to 15 ml of ice-cooled NaHCO₃ solution. The layers were separated and the organic phase was evaporated. The crude product was purified by column chromatography (20 g of silica gel, hexane:ethyl acetate=3:1) and the expected product was obtained as a light yellow oil. Yield: 410 mg (1.23 mmol, 31.4%).

### Synthesis of 4-(3-Fluoroprop-1-yn-1-yl)benzene-1,2-dicarboxylic acid (UB-12573)

To a DCM solution (8 ml) of 410 mg (1.23 mmol, 1 equiv.) UB-12572, 4 ml of TFA was added, and the mixture was stirred for 2 hours. TLC showed complete reaction. The reaction mixture was evaporated and the residue was suspended in 3 ml of heptane. The suspension was filtered, washed with heptane and dried. A pale brown solid was obtained.

Yield: 275 mg (1.23 mmol, 100%).

### Synthesis of [³H]APO-630

UB-12573 (3 mg) was dissolved in 3 ml of ethanol and 2.5 mg of 10% Pd/C catalyst was added. The mixture was reacted with tritium gas for 4 hours. The catalyst was filtered off, and the mixture was evaporated. To the crude material ethanol was added and evaporated again to eliminate mobile tritium. The residue was redissolved in ethanol and purified by HPLC.

The collected product fractions of five preparative injections were carefully evaporated, redissolved in 5 ml (measured by weight) of HPLC-grade ethanol and the total activity was measured and found to be 6.6 mCi. The solution was gravimetrically diluted to 6.6 ml (1.0 mCi/ml).

Replacement of tritiated methyl iodide by cold methyl iodide will lead to a cold analog, APO-630.

### Example 5

### Synthesis of 2-(4-(fluoromethyl)phenethyl)-2-methylmalonic acid (an exemplary embodiment of a compound of formula V)

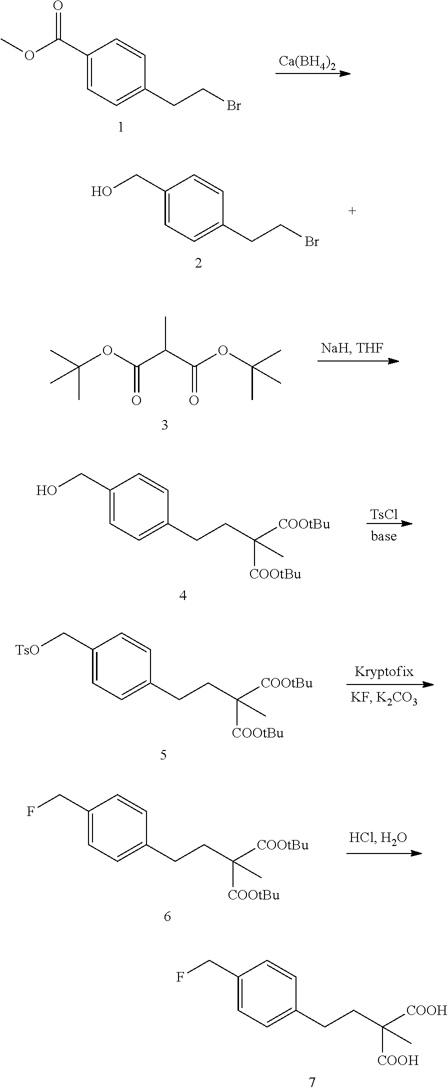

Other compounds may be used as starting compounds in a similar synthesis to obtain other embodiments of compounds of formula V.

### Example 5a

### Synthesis of {2-[4-(Methyl)phenyl]1,1,2,2-tetratritium-ethyl}-2-methylpropanedioic acid (compound of formula Va)

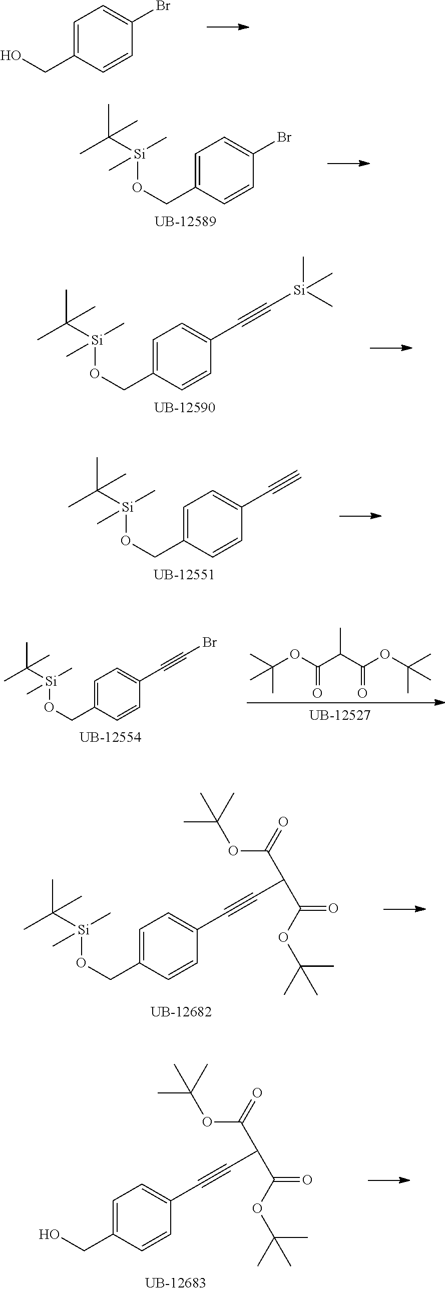

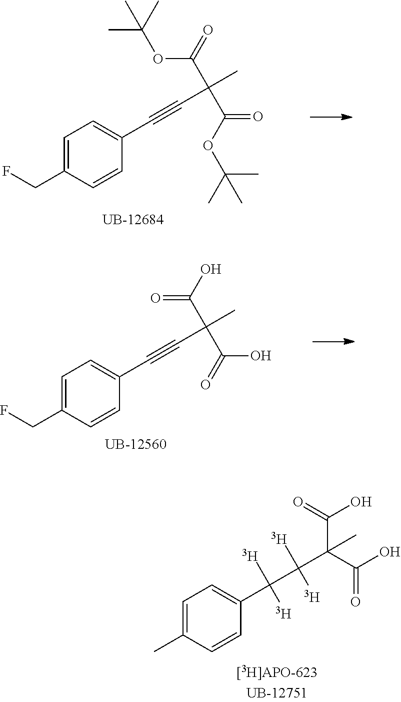

### Synthesis of [(4-Bromophenyl)methoxy](tert-butyl)dimethylsilane (UB-12589)

In an argon atmosphere, 20.0 g (107 mmol, 1 equiv.) of 4-bromobenzyl alcohol was dissolved in 80 ml of DCM. The solution was immersed into a water bath and stirred at room temperature. 14.6 g (214 mmol, 2 equiv.) of imidazole was added to the solution followed by tert-butyl dimethylchlorosilane (16.1 g, 107 mmol, 1 equiv., dissolved in 40 ml DCM) which was added dropwise. After 1 hour, the reaction was completed by GC. The reaction mixture was washed with 100 ml of water, then 100 ml of 1M HCl solution, 100 ml of saturated NaHCO₃ solution and finally 100 ml of brine. The organic solution was dried over Na₂SO₄ and evaporated. A thin, yellow oil was obtained. Yield: 31.5 g (104.5 mmol, 97.6%).

### Synthesis of [2-(4-{[(tert-Butyldimethylsilyl)oxy]methyl}phenyl)ethynyl]trimethylsilane (UB-12590)

31.5 (104.5 mmol, 1 equiv.) of UB-12589 was dissolved in 120 ml of triethylamine. To the solution 0.4 g (2.1 mmol, 0.02 equiv.) of copper(I) iodide and 2.4 g (2.1 mmol, 0.02 equiv.) of tetrakis(triphenylphosphine)palladium(0) was added, and the reaction mixture was degassed followed by inertization with argon. 15.2 ml (109.7 mmol, 1.05 equiv.) of TMS-acetylene was added to the solution in one portion. The resulting brown suspension was immersed into a preheated oil bath (70° C.). After 1 hour, the reaction was sampled and no reaction was observed by GC. After stirring overnight at 70° C., the reaction was assessed again and found to be completed. The reaction mixture was cooled to room temperature, and filtered through a pad of Celite. The pad was washed with 100 ml of methanol. The solutions were combined, evaporated, and redissolved in 100 ml of tert-butyl methyl ether (MTBE). The solution was filtered (to eliminate inorganic salts) and evaporated. The crude material was used in the next synthetic step without any purification.

### Synthesis of tert-Butyl[(4-ethynylphenyl)methoxy]dimethylsilane (UB-12551)

Crude UB-12590 was dissolved in 100 ml of methanol and to the solution 1 g of K₂CO₃ was added. The mixture was stirred at ambient temperature for 1 hour and then sampled. The cleavage of the TMS protecting group was completed by GC. The reaction mixture was filtered through a pad of Celite, and the filtrate was clarified with silica gel and activated carbon. The material was further purified using column chromatography (36 g of silica gel, n-hexane:toluene=4:1). Yield: 14.5 g GC: 86.4%.

### Synthesis of {[4-(2-Bromoethynyl)phenyl]methoxy}(tert-butyl)dimethylsilane (UB-12554)

14 g of UB-12551 from batch SI-1468.12 was dissolved in 85 ml of dry acetone. To the mixture 0.33 g (1.95 mmol, ˜0.04 equiv.) of AgNO₃ was added. The reaction mixture was stirred at room temperature under an argon atmosphere, 9.57 (53.74 mmol, ˜1.1 equiv) of N-bromosuccinimide was added in five portions over 50 minutes, and the mixture was stirred then for an additional 90 minutes. The reaction was sampled and it was found to be complete by TLC.

The reaction mixture was poured into 450 ml of heptane and stirred for 10 minutes. The resulting slurry was filtered and the filtrate was clarified with silica gel. Yield: 18.1 g (crude product, >100%).

### Synthesis of 1,3-Di-tert-butyl 2-methylpropanedioate (UB-12527)

5.0 g (23 mmol, 1 equiv.) of di-tert-butyl malonate was dissolved in 25 ml of DCM. 25 ml 50% NaOH solution and 785 mg (2.3 mmol, 0.1 equiv.) of tetrabutylammonium hydrogensulfate were added to the solution. 1.6 ml (25.4 mmol, 1.1 equiv.) of methyl iodide was added in one portion to the stirred mixture and the mixture was stirred at room temperature overnight. The reaction was assessed by GC and found to be incomplete. An additional 0.2 ml of methyl iodide was added, and after 3 hours, the next reaction check showed complete consumption of the starting di-tert-butyl malonate. 40 ml of water were added to the ice-cooled reaction mixture, the phases were separated, and the aqueous layer was extracted with 2×25 ml of DCM. The combined organic phases were washed with water and brine and then evaporated. During the evaporation, formation of crystals was observed and therefore the material was redissolved in 20 ml of MTBE. The solution was washed with 20 ml of water then the aqueous phase was back-extracted with 2×20 ml of MTBE. The combined organics were evaporated. Yield: 5.0 g (˜18.2 mmol, ˜79.12%)

### Synthesis of 1,3-Di-tert-butyl 2-[2-(4-{[(tert-butyldimethylsilyl)oxy]methyl}phenyl)-ethynyl]-2-methylpropanedioate (UB-12682)

5.0 g (˜18.2 mmol, 1.5 equiv.) of UB-12527 in 15 ml of dry THF, 9.1 ml of sodium bis(trimethylsilyl)amide (18.2 mmol, 1.5 equiv., 2M in THF) were added dropwise and the mixture stirred for 30 minutes at room temperature. A solution of UB-12554 in 10 ml of dry THF was added to the mixture over 15 minutes. The reaction was monitored by TLC. After 3.5 hours (˜70% conversion by TLC) the reaction was quenched by addition of 60 ml of saturated NH₄Cl solution. The organic layer was separated and the aqueous layer was washed with 3×20 ml of MTBE. The combined organic phases were washed with brine and dried over MgSO₄ and evaporated. The crude product was purified by column chromatography on 21 g of silica gel, eluted with toluene. Yield: 3.5 g Identified by ESI-MS ([M+Na]+: 497).

### Synthesis of 1,3-Di-tert-butyl 2-{2-[4-(hydroxymethyl)phenyl]ethynyl}-2-methylpropanedioate (UB-12683)

UB-12682 (3.5 g) was dissolved in 10 ml of dry THF and the flask was inerted with argon. 7.4 ml (7.4 mmol, ˜1 equiv.) of tetrabutylammonium fluoride (1M in THF) was added to the solution, and the room temperature cleavage of TBDMS protecting group was monitored by TLC. The reaction was complete in 2.5 hours. The solution was concentrated, and partitioned between 25 ml of MTBE and 10 ml of water. The organic phase was washed additionally with 3×10 ml of water and 10 ml of brine. The organic solution was dried over MgSO₄ and evaporated. Yield: 2.3 g (6.4 mmol).

### Synthesis of 1,3-Di-tert-butyl 2-{2-[4-(fluoromethyl)phenyl]ethynyl}-2-methylpropanedioate (UB-12684)

UB-12683 (1.5 g, 4.2 mml, 1 equiv.) was dissolved in dry DCM and the solution was cooled to below −70° C., and 0.8 ml (6.2 mmol, 1.5 equiv.) of DAST dissolved in 2 ml of DCM was added. The temperature was maintained below −70° C. for an hour, then the reaction was sampled and checked by TLC. The conversion of the starting material was complete. The reaction mixture was poured into 50 ml of ice-cooled Na₂CO₃, the phases were separated and then the aqueous layer was extracted with 2×20 ml of DCM. The organics were combined and evaporated, and the crude product was purified using column chromatography on 30 g silica gel (hexane:ethyl acetate=10:1). Yield: 250 mg (0.69 mmol, 16.7%).

### Synthesis of 2-{2-[4-(Fluoromethyl)phenyl]ethynyl}-2-methylpropanedioic acid (UB-12560)

200 mg of UB-12684 was dissolved in 2 ml of TFA, the reaction stirred for one hour and found to be complete by TLC. The sample was evaporated to yield a brown oil. Crude UB-12560 was analyzed by LC-MS, and the peak at 18.80 was identified as the expected product ([M−H]: 249.

### Synthesis of [3H]APO-623

UB-12560 (7.8 mg) was dissolved in 3 ml of ethanol and 3 mg of 10% Pd/C catalyst was added. The mixture was reacted with tritium gas for three hours. The catalyst was filtered off, and the mixture was evaporated. Ethanol was added to the crude material and evaporated again to eliminate mobile tritium. The residue was redissolved in ethanol and purified by HPLC. The collected product fractions of four preparative injections were carefully evaporated, redissolved in 3 ml (measured by weight) of HPLC-grade ethanol and the total activity was measured and found to be 6.5 mCi. The solution was gravimetrically diluted to 6.5 ml (1.0 mCi/ml).

Replacement of tritium gas by hydrogen gas will lead to cold analog, APO-623.

### Example 5b

### Synthesis of 2-tritritiummethyl[5-(2-nitro-1H-imidazol-1-yl)pentyl]propanedioic acid (compound of formula Vd)

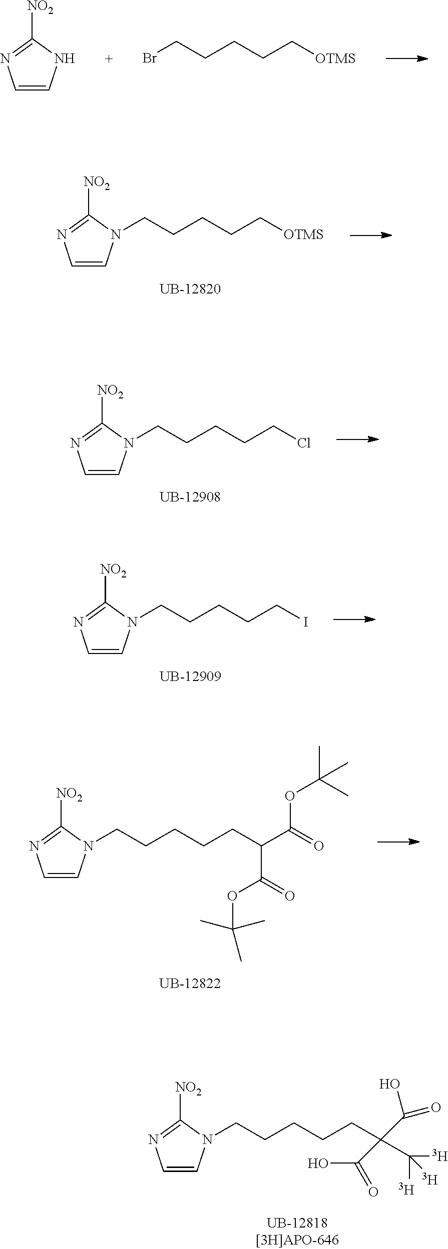

### Synthesis of 2-Nitro-1-{5-[(trimethylsilyl)oxy]pentyl}-1H-imidazole (UB-12820)

2-Nitroimidazole (2.0 g, 17.7 mmol, 1 equiv.) was dissolved in 170 ml of acetonitrile and 40 ml of dimethylformamide. [(5-bromopentyl)oxy]trimethylsilane (UB-12819, 7.46 g, 31.0 mol, 1.75 equiv.) and 7.37 g (53.07 mmol, 3.0 equiv.) of potassium carbonate were added to the solution. The reaction mixture was stirred at 60° C. for 24 hours. IPC (TLC, hexane:acetone=1:1) showed complete reaction. The reaction mixture was filtered, and the filtrate was evaporated. The crude product was purified by flash chromatography. Yield: 3.06 g (11.26 mmol, 63.6%) of light yellow oil. Identification: ESI-MS ([M+H+]=272, [M+Na+]=294).

### Synthesis of 1-(5-Chloropentyl)-2-nitro-1H-imidazole (UB-12908)

The title compound was formed as a by-product via deprotection of UB-12820 and reaction of the formed alcohol with tosyl chloride. The tosylate was formed just as a minor component. 606 mg (2.23 mmol, 1.0 equiv.) of UB-12820 was dissolved in 10 ml tetrahydrofuran and 6.7 ml (3 equiv.) tetra-n-butylammoniumfluoride (1M solution in tetrahydrofuran) was added. The mixture was stirred at ambient temperature for 4 hours and was monitored by TLC. After completion the tetrahydrofuran was evaporated, 10 ml of dichloromethane was added and was washed 4 times with 10 ml water. The first aqueous phase was extracted twice with 10 ml dichloromethane. The organic phases were combined and the solvent was evaporated. Three time 10 ml isopropanol then 5 ml of methanol was evaporated from the product. 510 mg product was isolated as a yellow oil. The oil was dissolved in 20 ml of dichloromethane, 589 mg (3.1 mmol, 1.4 equiv.) of p-toluenesulfonyl chloride and 720 μl (5.1 mmol, 2.3 equiv.) of triethylamine was added to the solution and was stirred at ambient temperature. TLC showed the reaction is progressing slowly. After 5 hours the temperature was raised to 37° C. After an overnight stirring, the reaction was not complete but the TLC showed multiple components so it was worked up. The reaction mixture was washed four times with 10 ml brine. The organic phase was dried over sodium sulphate. The solvent was evaporated, the crude product was fractionated by flash chromatography. The process was repeated using 1.4 g of UB-12820. The isolated products were combined, 338 mg of UB-12821 and 531 mg of UB-12908 was isolated, and the latter was selected as starting material for synthesis of UB-12909. Yield (UB-12908): 33.2%. Identification: ESI-MS ([M+H+]=218, [M+Na+]=240, [M+MeOH+Na+]=272, [2M+Na+]=457).

### Synthesis of 1-(5-Iodopentyl)-2-nitro-1H-imidazole (UB-12909)

UB-12908 (531 mg, 2.44 mmol) was dissolved in 20 ml acetone and 718 mg sodium iodide (4.8 mmol, 2 equiv.) was added. The mixture was stirred at 58° C. for 24 hours. The solid was filtered and washed with acetone and was measured to determine the conversion. The conversion was about 60% thus 359 mg sodium-iodide (2.4 mmol, 1 equiv.) was added and stirring was continued for 24 hours at 58° C. The reaction mixture was filtered, the solvent was evaporated. The residue was washed with 4×5 ml dichloromethane and was filtered. The solvent was evaporated. Identification: ESI-MS ([M+Na+]: 332, [M+Na+CH3OH+]: 364, [2M+Na+]: 641).

### Synthesis of 1,3-Di-tert-butyl 2-[5-(2-nitro-1H-imidazol-1-yl)pentyl]propanedioate (UB-12822)

Di-tert-butyl malonate (797 mg, 3.68 mmol, 2.0 equiv.) and 216 mg (2.25 mmol, 1.2 equiv.) of sodium tert-butoxide were dissolved in 15 ml THF at 0° C. After 1 hour stirring 569 mg UB-12909 (1.85 mmol, 1.0 equiv.) was added in 4 ml THF at 0° C. The mixture was stirred for 2 hours. IPC TLC (n-hexane:EtOAc=1:1) showed complete conversion. The reaction mixture was quenched by addition of 2N HCl solution (pH-1), then THF was removed by rotary evaporation. 10 ml of ethyl acetate was added to the residue, the water phase was separated and the organic phase was washed with water and brine. The organic phase was dried over sodium sulphate, filtered and evaporated. The crude product was purified by flash chromatography to yield 350 mg (0.88 mmol, 47.6%) of UB-12822. Identification: ESI-MS ([M+Na+]: 420, [M+K+]: 436, [2M+Na+]: 817, [2M+K+]: 833).

### Synthesis of [3H]APO-646

Into a 4 ml sample vial 19 mg (0.2 mmol) of sodium tert-butoxide and 3 ml of THF solution of UB-12822 (17.5 mg/ml, 0.13 mmol) was weighed in. The vial was flushed with argon, closed and the mixture was stirred for 30 minutes without cooling. A 0.1 ml aliquot of this reaction mixture (˜4.3 μmol of deprotonated UB-12822) was transferred into a 0.2 ml sample vial, and 100 mCi of methyl iodide (˜1.3 μmol, in 0.1 ml of toluene) was added. The reaction mixture was stirred for 20 hours at room temperature. The mixture was evaporated by a gentle steam of nitrogen, and to the residue 0.1 ml of trifluoroacetic acid was added. After 6 hours the reaction was found to be complete (HPLC). The reaction mixture was evaporated and the residue was dissolved in 10 ml of ethanol. Activity of the crude product: 30 mCi. The crude product was purified by HPLC.

Replacement of tritiated methyl iodide by cold methyl iodide will lead to cold analog.

### Example 5c

### Synthesis of 2-methyl-2-{4-[(5-nitro-1,3-thiazol-2-yl)tritritiummethylamino]butyl}propanedioic acid (compound of formula VI)

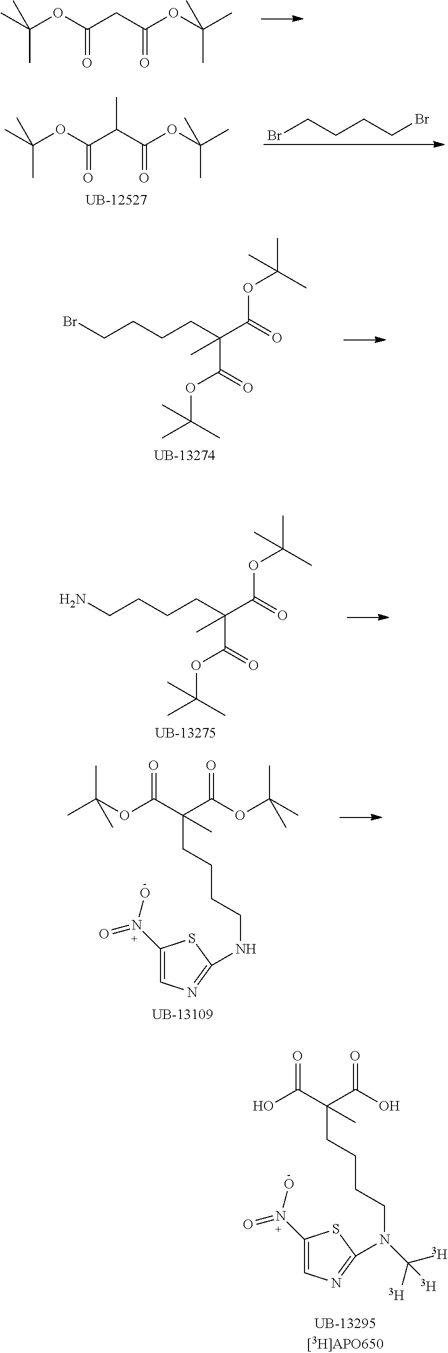

### Synthesis of 2-Methyl-di-tert-butyl malonate (UB-12527)

Di-tert-butyl malonate (40 g, 184.9 mmol, 1.0 equiv.) was dissolved in 200 ml of dichloromethane. To the solution 7.2 g (18.5 mmol, 0.1 equiv.) of tetrabutylammonium hydrogensulfate and a solution of sodium hydride (100 g in 200 ml of water) was added to the cloudy, stirred mixture 12.7 ml (203.5 mmol, 1.1 equiv.) of methyl iodide was added dropwise, without cooling. Progression of the reaction was monitored by GC. Additional amount of methyl iodide was added in portions (altogether 3.75 ml, 61 mmol, 0.33 equiv.) to complete the reaction. The mixture was diluted with 300 ml of water, the organic phase was separated, and the water phase was extracted with dichloromethane (3×200 ml). Combined organics were washed with water and brine, dried and evaporated. The residue was redissolved in 200 ml of tert-butyl methyl ether (MTBE), washed with 200 ml of water, dried over sodium sulfate and evaporated. Yield: 40.36 g (175 mmol, 94.7%) of dark yellow oil. GC: 80.6%

### Synthesis of 1,3-Di-tert-butyl 2-(4-bromobutyl)-2-methylpropanedioate (UB-13274)

To a salted ice (−10-0° C.) cooled solution of UB-12527 (20.0 g, 86.8 mmol, 1.0 equiv., in 190 ml of dry tetrahydrofuran) 3.47 g (86.8 mmol, 1.0 equiv., 60% in mineral oil) of sodium hydride was added. The mixture was warmed to room temperature then stirred for 30 min. To the resulting white suspension 15.5 ml (130 mmol, 1.5 equiv.) of 1,4-dibromobutane was added and the mixture was stirred overnight. The mixture was evaporated and the residue was dissolved in 250 ml of water and 250 ml of MTBE. The phases were separated, the organic was dried over sodium sulfate and evaporated. The crude product was purified by rectification (0.7-0.8 mbar, 111-125° C.). Yield: 14.4 g (39.5 mmol, 45.5%) colourless, clear oil. GC: 96.7%.

### Synthesis of 1,3-Di-tert-butyl 2-(4-aminobutyl)-2-methylpropanedioate (UB-13275)

UB-13274 (7.0 g, 19.2 mmol) was dissolved in 70 ml of dimethylsulfoxide and 2.5 g sodium azide (38.3 mmol, 2.0 equiv.) was added. The mixture was stirred at room temperature and the reaction was complete after 3 hours (GC). The reaction mixture was poured into 700 ml of water then the solution was extracted with 3×140 ml of diethyl ether. The combined organics were concentrated to ˜80 ml. To the residue 100 ml of THF, 10.0 g (38.3 mmol, 2.0 equiv.) of triphenylphosphine and 6 ml of water was added. The reaction mixture was stirred overnight. The reaction was complete by TLC (methanol:dichloromethane=1:1, visualization: ninhydrin), the mixture was evaporated and dried by repeated evaporation of 30 ml of ethanol. The crude product was purified by column chromatography on 200 g of silica gel, eluent: EtOAc:methanol=1:1. Yield: 5.15 g (17.08 mmol, 89.2%) opalescent oil. Identification: ESI-MS ([M+]: 302, [M+Na+]: 324).

### Synthesis of 1,3-Di-tert-butyl 2-methyl-2-{4-[(5-nitro-1,3-thiazol-2-yl)amino]butyl}propanedioate (UB-13109)

UB-13275 (1.0 g, 3.31 mmol, 1.0 equiv.) and 690 mg (3.31 mmol, 1.0 equiv.) of 2-bromo-5-nitrothiazole were dissolved in 10 ml of acetonitrile. To the solution N-ethyldiisopropylamine (1.16 ml, 6.62 mmol, 2.0 equiv.) was added, and the reaction mixture was stirred for 2 hours without heating. The reaction was incomplete by TLC (n-hexane:EtOAc=1:1). To the mixture additional 0.2 g of UB-13275 was added and the reaction was stirred for 2 hours. TLC showed complete conversion of 2-bromo-5-nitrothiazole. The reaction mixture was evaporated and the expected product was isolated by column chromatography (50 g silica gel, n-hexane:EtOAc=2:1). Yield: 550 mg (1.28 mmol, 31.7%). Identification: ESI-MS ([M+H+]: 430, [M+Na+]: 452, [2M+Na+]: 881).

### Synthesis of [3H]APO-650

In 1.0 ml of dry dimethyl formamide (DMF) 10.0 mg of UB-13109 was dissolved. Into a 4 ml sample vial 0.2 ml of the DMF solution (˜0.0047 mmol, 4 equiv.) and 2 mg of potassium carbonate was weighed in. To the mixture 100 mCi of methyl iodide (500 mCi/ml in toluene, ˜0.0013 mmol, 1 equiv.) was added, the vial was closed immediately, and the mixture was stirred for 3 hours at room temperature. The mixture was evaporated and the residue was dissolved in 1 ml of dichloromethane and 0.2 ml of trifluoroacetic acid. After an overnight stirring at room temperature the reaction mixture was sampled and analyzed by HPLC. Formation of the expected product was observed at 10.09 min. The reaction mixture was evaporated and the residue was dissolved in 0.2 ml of methanol. The crude product was purified by HPLC.

Replacement of tritiated methyl iodide by cold methyl iodide will lead to cold analog.

### Example 5d

### Synthesis of 2-methyl-2-{4-[(5-nitro-1,3-thiazol-2-yl)tritritiummethylamino]butyl}propanedioic acid (compound of formula Vi)

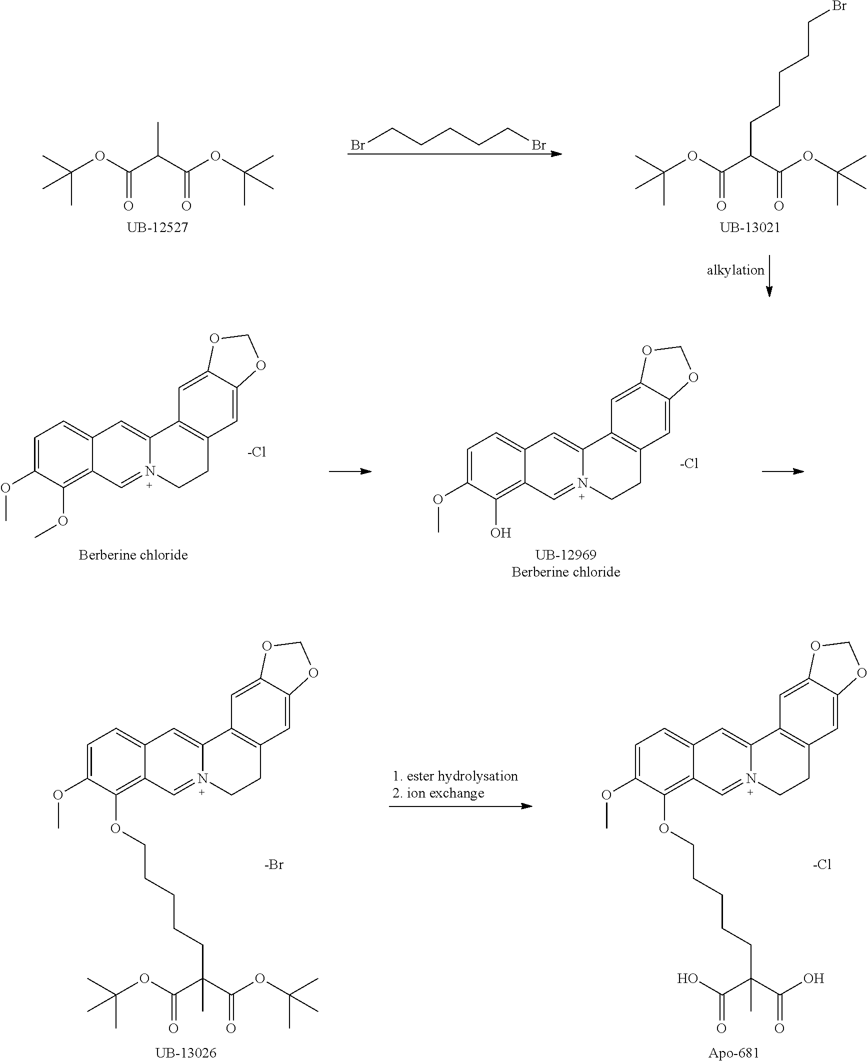

### Synthesis of 1,3-Di-tert-butyl 2-methylpropanedioate (UB-12527)

A 1 L three-necked flask was charged with 40.0 g (184.9 mmol, 1.0 eq.) di-tert-butyl-malonate, 200 ml dichloromethane, 7.2 ml (18.5 mmol, 0.1 eq.) tetrabutylammonium hydrogen sulfate and 200 ml solution of 50% NaOH in water. To the resulting biphasic mixture 12.7 ml (203.4 mmol, 1.1 eq.) iodomethane was added at room temperature and the reaction mixture was stirred for 4 hours at room temperature. Monitoring of the reaction: a small sample of the well-stirred reaction mixture was added to a mixture of water and EtOAc and the organic phase was analyzed by GC. Into the reaction mixture water (300 ml) was added dropwise at 10° C. The layers were separated and the aqueous water was extracted with dichloromethane (2×200 ml). The combined organic phases was washed with brine (200 ml), dried over Na₂SO₄ and evaporated to dryness to give 35.6 g UB-12527 as a yellow oil. Yield of product: 35.6 g (154.6 mol, 83.6%) yellow oil. Analysis: GC: 89.0%

### Synthesis of Berberrubine chloride (UB-12969)

30.0 g (80.7 mmol, 1.0 eq.) berberine chloride hydrate was heated under vacuum (1.0 mbar) in neat at 190° C. for 1 hour. The crude product was recrystallized from ethanol (80 ml). Yield: 24.8 g (69.3 mol, 85.9%) red solid

### Synthesis of 1,3-Di-tert-butyl 2-(5-bromopentyl)-2-methylpropanedioate (UB-13021)

A 1 liter three-necked flask was charged with 32.0 g (138.9 mmol, 1.0 eq) UB-12527, 300 ml dry tetrahydrofurane. The solution was cooled to 0° C. and 5.6 g (138.9 mmol, 1.0 eq.) NaH was added in small portions. After addition the reaction mixture was stirred for 30 minutes at room temperature then 48.0 g (208.4 mmol, 2.0 eq) 1,5-dibromopentane was added.

The reaction mixture was stirred at room temperature overnight. Monitoring of the reaction: a small sample of the well-stirred reaction mixture was added to a mixture of water and MTBE and the organic phase was analyzed by GC. The reaction mixture was evaporated to dryness and the residue was dissolved in MTBE (400 ml), washed with water (250 ml) and brine (250 ml), dried over Na₂SO₄ and evaporated to dryness to give 73.6 g UB-13021 as a yellow oil. The crude product was purified by vacuum distillation (125-145° C., 1.7 mbar). Yield of product: 31.7 g (82.2 mmol, 59.1%) yellow oil. Analysis: GC: 96.2%.

### Synthesis of 16-{[7-(Tert-butoxy)-6-[(tert-butoxy)carbonyl]-6-methyl-7-oxoheptyl]oxy}-17-methoxy-5,7-dioxa-13-lambda{5}-azapentacyclo[11.8.0.0{2,10}.0{4,8}.0{15,20}]henicosa-1(21),2(10),3,8,13,15,17,19-octaen-13-ylium bromide (UB-13026)

A 250 ml three-necked flask was charged with 10.0 g (27.9 mmol, 1.0 eq) berberrubine chloride, 100 ml dry DMF and 15.9 g (41.9 mmol, 1.5 eq) UB-13021 and the reaction mixture was stirred at 100° C. for 2 hours. Monitoring of the reaction: a small sample of the well-stirred reaction mixture was added into methanol and analyzed by TLC (silica plate, eluent was dichloromethane/methanol=85/15). The reaction mixture was evaporated to dryness, the residue was suspended in diethyl ether, filtered and the crude product (21 g) was purified by column chromatography (600 g silica gel, 5 L eluent (dichloromethane/MeOH=85/15)). Yield: 14.0 g (20.6 mmol, 73.8%) yellow solid. Analysis: HPLC: 97.1%.

### Synthesis of APO-681

A 500 mL three-necked flask was charged with 10.0 g UB-13026 (14.3 mmol), 100 ml dichloromethane and 50 ml trifluoroacetic acid. The brown solution was stirred at room temperature for 1 hour. Monitoring of the reaction: a small sample of the aqueous phase of the well-stirred reaction mixture was analyzed by TLC (silica plate, eluent was dichloromethane/methanol=85/15). The reaction mixture was evaporated to dryness to give 13.0 g brown oily product. This crude product was dissolved in a mixture of ethanol (400 ml) and water (200 ml) and stirred with ion exchange resin (Amberjet 4200, Cl-form) overnight. The color of the solution turned to yellow. The resin was removed by filtration and washed with ethanol and water subsequently. The filtrate was evaporated resulting in 6.5 g yellow solid with 94.7% purity (HPLC). The crude product was chromatographed on silica (150 g) using dichloromethane/MeOH=85/15 as eluent (3.0 L) to give 5.2 g yellow solid with 95.8% purity. Yield: 5.2 g (9.6 mmol, 67.0%) yellow solid. Analysis: HPLC: 95.8%

¹H NMR [Bruker Avance 500 (500 MHz, DMSO, TMS as internal standard]: δ 1.22 (s, 3H, CH₃), 1.24 (t, 2H, CH₂), 1.44 (t, 2H, CH₂), 1.70 (t, 2H, CH₂), 1.84 (t, 2H, CH₂), 3.19 (t, 2H, CH₂), 4.05 (s, 3H, OCH₃), 4.27 (t, 2H, OCH₂), 4.97 (t, 2H, CH₂), 6.17 (s, 2H, O—CH₂—O), 7.08 (s, 1H, Ar—H), 7.78 (s, 1H, Ar—H), 7.97 (d, 1H, Ar—H), 8.18 (d, 1H, Ar—H), 8.92 (s, 1H, Ar—H), 9.73 (s, 1H, Ar—H).

Identification: ESI-MS ([M+H+]=508, [M+Na+]=530).

### Example 5e

### Synthesis of 2-methyl-2-(5-{[(10S)-3,4,5,14-tetramethoxy-13-oxotricyclo[9.5.0.0{2,7}]hexadeca-1(16),2,4,6,11,14-hexaen-10-yl]carbamoyl}pentyl)propanedioic acid (compound of formula Vk)

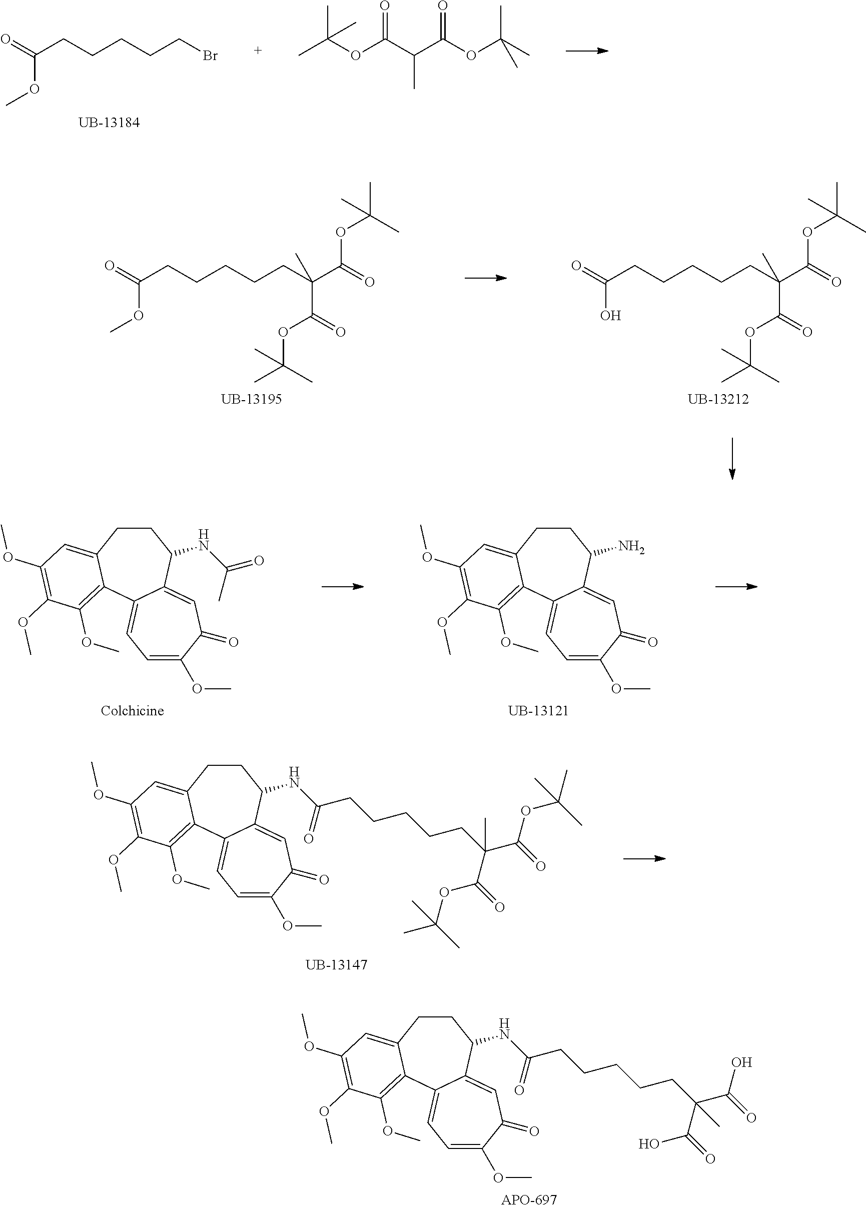

### Synthesis of 1,1-Di-tert-butyl 6-methyl 1-methylhexane-1,1,6-tricarboxylate (UB-13195)

A 100 mL three-necked flask was charged with 5.7 g (17.8 mmol, 1.0 eq.) 2-methyl di-tert-butyl malonate, 30 ml dry tetrahydrofurane and the resulting solution was cooled to 0° C. Into the well stirred reaction mixture 783 mg (19.6 mmol, 1.1 eq.) of sodium hydride were added in portions at 0° C. After the addition the reaction mixture was allowed to warm to room temperature and a solution of 3.7 g (17.8 mmol, 1.0 eq.) 6-bromohexanoic acid methyl ester dissolved in tetrahydrofurane (10 ml) was added and the reaction mixture was refluxed overnight. Monitoring of the reaction: a small sample of the well-stirred reaction mixture was added into a mixture of water and ethyl acetate and the organic phase was analyzed by TLC (silica plate, eluent was hexane/MTBE=5/1). The reaction was complete. Into the reaction mixture water (30 ml) and ethyl acetate (50 ml) was added. The layers were separated and the aqueous phase was extracted with ethyl acetate (2×50 ml). The combined organic phases was washed with brine (50 ml), dried over Na₂SO₄ and evaporated to dryness. The crude product (7.6 g) was purified by vacuum distillation (128-132° C./0.9 mbar). Yield of product: 2.7 g (7.5 mol, 42%) colorless oil. Analysis: GC: 98.3%

### Synthesis of 8-(tert-butoxy)-7-[(tert-butoxy)carbonyl]-7-methyl-8-oxooctanoic acid (UB-13212)

A 100 mL three-necked flask was charged with 2.7 g (7.5 mmol, 1.0 eq.) UB-13195 and 30 ml methanol. In to this mixture a solution of 0.9 g (37.7 mmol, 5.0 eq) lithium hydroxide in water (15 ml) was added drop-wise maintaining the inner temperature below 10° C. After addition the reaction mixture was stirred at room temperature for 1 hour. Monitoring of the reaction: a small sample of the well-stirred reaction mixture was added into a mixture of phosphoric acid solution and ethyl acetate and the organic phase was analyzed by TLC (silica plate, eluent was hexane/ethyl acetate=10/1). The reaction was completed.

The reaction mixture was evaporated and the residue was dissolved in water (20 ml), the pH was adjusted to 3-4 with phosphoric acid solution (85% in water, 5 ml) and extracted with EtOAc (2×30 ml). The organic layer was washed with brine, (20 ml) dried over Na₂SO₄ and evaporated to dryness. Yield of product: 2.5 g (7.4 mol, 99%) colorless oil. Analysis: GC: 91.3%.

### Synthesis of N-Desacetylcolchicine (UB-13121)

1^{st} step: A 250 mL three-necked flask was charged with 9.0 g (26.0 mmol, 1.0 eq.) colchicine, 90 ml acetonitrile, 2.8 g (26.0 mmol, 1.0 eq.) 4-dimethylaminopyridine, 6.3 ml (52.0 mmol, 2.0 eq.) triethylamine and 11.8 g (62.0 mmol, 2.4 eq.) di-tert-butyl dicarbonate and the reaction mixture was stirred at 100° C. for 3 hours. Then 11.4 g (52.0 mmol, 2.0 eq.) di-tert-butyl dicarbonate was added and the reaction mixture was stirred further 2 hours at 100° C. Monitoring of the reaction: a small sample of the well-stirred reaction mixture was added into a mixture of citric acid solution and ethyl acetate and the organic phase was analyzed by TLC (silica plate, eluent was hexane/ethyl acetate=1/4). Approx. 10% starting material and 90% product was detected. The reaction mixture was concentrated to a half volume and chloroform (300 ml) was added. The resulting solution was washed with 5% solution of citric acid (3×100 ml). The organic phase was dried over Na₂SO₄ and evaporated to dryness to give 17.8 g dark brown oil.

2^{nd} step: The product of the first step was dissolved in 27 ml methanol and 0.9 ml (26.0 mmol, 1.0 eq.) 30% solution of sodium methoxide in methanol was added drop-wise at room temperature. The given dark brown solution was stirred for 1 hour. Monitoring of the reaction: a small sample of the well-stirred reaction mixture was analyzed by TLC (silica plate, eluent was hexane/ethyl acetate=1/4). Approx. 20% Colchicine and 70-75% product was detected. Brine (150 ml) was added to the reaction mixture and the aqueous solution was extracted with dichloromethane (3×50 ml). The organic layer was dried over Na₂SO₄ and evaporated to dryness to give 12.7 g brown foam.

3^{rd} step: The product of the second step was dissolved in 18 ml trifluoroacetic acid at room temperature and stirred for 15 minutes. Monitoring of the reaction: a small sample of the well-stirred reaction mixture was analyzed by TLC (silica plate, eluent was methyl ethyl ketone/acetic acid/water=16/3/2.5). Approx. 25% Colchicine and 70-75% product was detected. The reaction mixture was evaporated, the residue was taken up in 5% solution of citric acid (180 ml) and washed with dichloromethane (2×150 ml). The pH of the aqueous phase was adjusted to 10 with 10% solution of NaOH and then extracted with dichloromethane (3×180 ml). The organic phase was dried over Na₂SO₄ and evaporated to dryness to give 5.7 g brown foam. The crude product was purified by flash chromatography using methyl ethyl ketone/acetic acid/water=16/3/2.5 as eluent. Yield of product: 4.0 g (11.2 mol, 43%) yellow solid.

### Synthesis of 1,3-Di-tert-butyl 2-methyl-2-(5-{[(10S)-3,4,5,14-tetramethoxy-13-oxotricyclo[9.5.0.0{2,7}]hexadeca-1(16),2,4,6,11,14-hexaen-10-yl]carbamoyl}pentyl)propanedioate (UB-13147)

A 250 mL three-necked flask was charged with 2.33 g (6.8 mmol, 1.0 eq) UB-13212, 40 ml dry dichloromethane, 2.42 g (6.8 mmol, 1.0 eq., AB-1244.12) UB-13121, 0.91 g (6.8 mmol, 1.0 eq.) N-hydroxybenzotriazole hydrate and 1.42 g (7.4 mmol, 1.1 eq.) EDC and the reaction mixture were stirred overnight at room temperature. Monitoring of the reaction: a small sample of the well-stirred reaction mixture was added into a mixture of water and ethyl acetate and the organic phase was analyzed by TLC (silica plate, eluent was dichloromethane/methanol=9/1). The reaction was completed. The reaction mixture was diluted with dichloromethane (20 ml) and washed with water (30 ml), saturated NaHCO₃ solution (30 ml) and brine (30 ml). The organic phase was dried over Na₂SO₄ and evaporated to dryness to give 5.3 g brown foam. The crude product was purified by column chromatography on silica using dichloromethane/methanol=95/5 as eluent (800 ml). Yield of product: 2.10 g (3.1 mmol, 45%) red foam.

### Synthesis of APO-697

A 100 mL three-necked flask was charged with 2.1 g (3.1 mmol, 1.0 eq) UB-13147, 20 ml dichloromethane and 10 ml trifluoroacetic acid. The solution was stirred at room temperature for 2 hours. Monitoring of the reaction: a small sample of the well-stirred reaction mixture was analyzed by TLC (silica plate, eluent was dichloromethane/methanol=9/1). The reaction was completed. The reaction mixture was evaporated and the given dark brown oily residue was purified by column chromatography on silica (68 g) using dichloromethane/methanol=95/5 (700 ml) as eluent. Yield: 1.4 g (2.5 mmol, 81%) beige solid. Analysis: HPLC: 97.6%

¹H NMR [Bruker Avance 500 (500 MHz, DMSO, TMS as internal standard]: δ 1.18 (m, 6H, CH₂), 1.21 (s, 3H, CH₃), 1.42 (d, 2H, CH₂), 1.63 (t, 1H, CH), 1.65 (t, 1H, CH), 1.84 (s, 2H, CH₂), 2.10 (t, 1H, CH), 2.58 (t, 1H, CH), 3.53 (s, 3H, CH₃), 3.83 (s, 3H, CH₃), 3.87 (s, 3H, CH₃), 4.32 (s, 1H, CH—N), 6.76 (s, 1H, Ar—H), 7.01 (s, 1H, Ar—H), 7.10 (d, 2H, Ar—H), 8.48 (s, 1H, NH).

ESI-MS: [M+H]+=572, [M+Na]+=594

Apo-697 is the cold analog of Apo-698

### Example 6

### Synthesis of 5,5′(7-fluoroheptane-2,2-diyl)bis(1H-tetrazole) (an exemplary embodiment of a compound of formula VII)

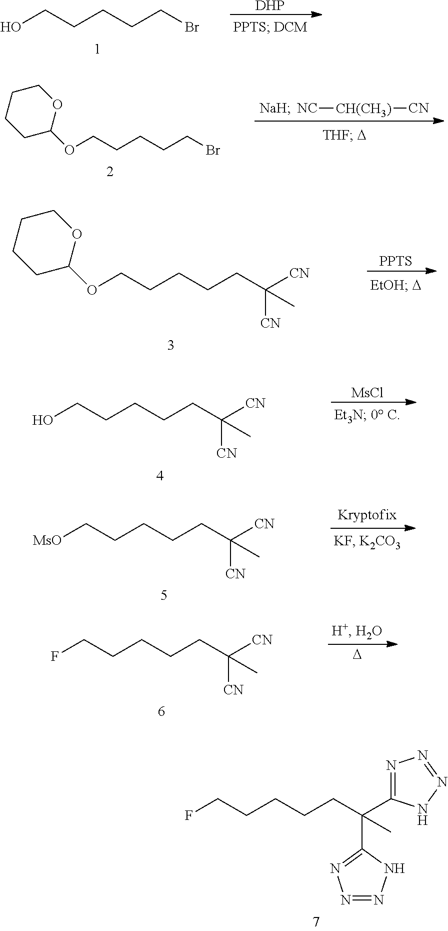

5-bromo-1-pentanol (1), 3 g, is treated with 1.5 eq of 3,4-dihydro-2H-pyran and 0.1 eq of pyridinium para toluenesulfonate (PPTS) in 135 mL of CH₂Cl₂. Other starting materials, such as bromo C₁₋₉ alcohols may be used to obtain other embodiments of compounds of formula VII.

After work-up and purification, 1.45 g (33%) of product 2 is obtained. 1.0 eq of methyl malonitrile is deprotonated with 1 eq of NaH and 1.0 eq of bromide 2 is added along with catalytic amount of KI at 50° C. A complete conversion can be observed after 10 hours and a 90% yield can be obtained. Deprotection of tetrahydro pyran (THP) can be done with PPTS in ethanol at 55° C. After work-up, a quantitative yield of alcohol 4 is obtained and directly used for the mesylation reaction. With the mesylate 5 in hand, a kryptofix-mediated fluorination can be performed. Compound 6 is obtained in 68% yield. 12 mL of DMF, 10 eq. of acetic acid, triethylamine and sodium azide each are added to compound 6. The resulting mixture is stirred at 140° C. for 19 hours. Water and 1N HCl are added. The solid is filtered off and washed with water. Compound 7 is obtained in a 60% yield.

¹H NMR [for example, Bruker Avance 400 (400 MHz, CDCl3, TMS as internal standard] of the 5,5′(7-fluoroheptane-2,2-diyl)bis(1H-tetrazole) compound shows the following results: δ 1.29 (m, 4H, CH₂), 1.49 (m, 2H, CH₂), 1.77 (s, 3H, Me), 1.87 (t, 2H, CH₂), 4.09 (m, 2H, CH₂F).

### Example 6a

### Synthesis of 1-fluoro-3,4-ditritium-6,6-di(1H-tetrazol-5-yl)heptane (compound of formula VIIa)

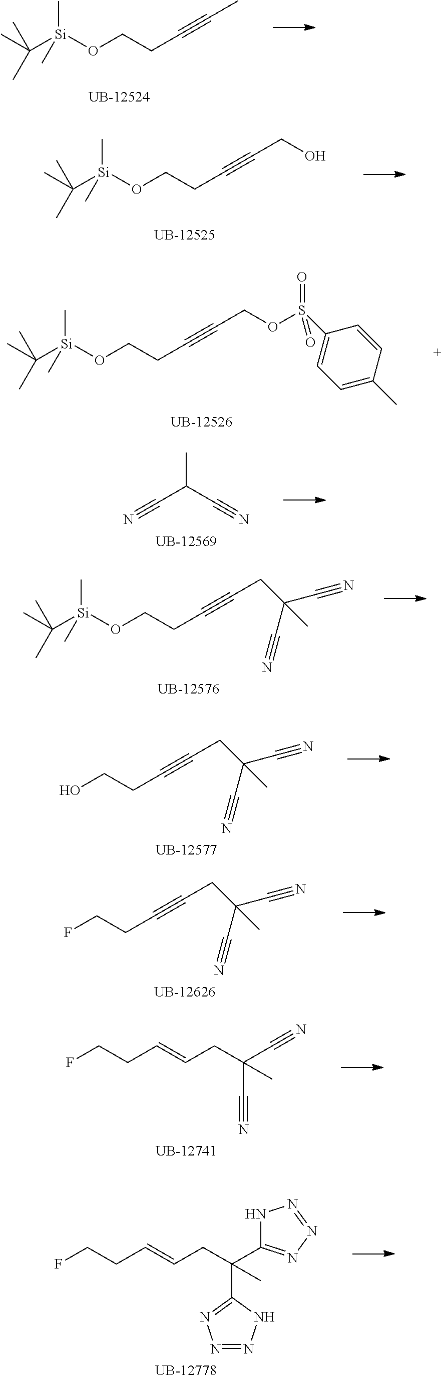

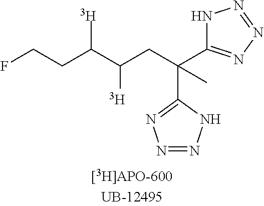 [SMILES: [3H]C(CCF)C([3H])CC(C)(c1nnn[nH]1)c1nnn[nH]1]

### Synthesis of (But-3-yn-1-yloxy)(tert-butyl)dimethylsilane (UB-12524)

30.26 g (428.0 mmol, 1 equiv.) 3-butyn-1-ol was dissolved in 500 ml THF. To this solution 71.34 g (1.027 mol, 2.4 equiv.) of imidazole and 77.95 g (513.5 mmol, 1.2 equiv.) of TBDMSCl was added and the reaction mixture was stirred vigorously for 5 hours. Then the mixture was passed through a short silica pad, evaporated and fractionated in vacuo, using a Vigreux column. Yield: 40.04 g (206.3 mmol, 48%) GC: 95%.

### Synthesis of 5-[(tert-Butyldimethylsilyl)oxy]pent-2-yn-1-ol (UB-12525)

40.04 g (207.6 mmol, 1 equiv.) of UB-12524 was dissolved in 490 ml of diethyl ether and was cooled to −78° C. To this solution 94.48 ml (236.2 mmol, 1.14 equiv.) of n-butyllithium (2.5M in hexane) was added followed by 31.42 g (1046.3 mmol, 5.04 equiv.) of paraformaldehyde. The acetone/dry ice bath was then removed and the mixture was warmed to room temperature.

After stirring overnight the reaction was quenched by addition of 200 ml of saturated ammonium chloride solution. THF was removed by rotary evaporation. The residue was extracted with diethyl ether (4×100 ml), the organic phases were combined, filtered through a pad of Celite, concentrated to 100 ml of volume, dried over Na₂SO₄ and evaporated. Yield: 4.06 g.

### Synthesis of 5-[(tert-Butyldimethylsilyl)oxy]pent-2-yn-1-yl 4-methylbenzene-1-sulfonate (UB-12526)

47.06 g (219.5 mmol, 1 equiv.) of UB-12525 and 50.77 g (263.4 mmol, 1.2 equiv.) of p-toluenesulfonyl chloride were dissolved in 450 ml of diethyl ether, cooled to −10° C. and 124.33 g (2.195 mol, 10 equiv.) of potassium hydroxide was added to the stirred reaction mixture in five portions. The mixture was warmed to room temperature and stirred for 4 hours. The reaction was complete by TLC. The reaction mixture was poured into 1300 ml of ice cold water. The organic phase was separated, the aqueous phase was then extracted with diethyl ether (5×100 ml). The organic phases were combined, filtered through a pad of Celite, dried over Na₂SO₄ and evaporated. Prepurification was carried out using a 500 g silica gel pad eluted with ˜2500 ml of hexane:acetone=4:1. Final purification was achieved using flash-chromatography. Yield: 26.60 g (72.17 mmol, 33%).

### Synthesis of 2-Methylpropanedinitrile (UB-12569)

30.0 g (454 mmol, 2 equiv.) of malononitrile was mixed by intensive stirring with 14.1 ml (226.8 mmol, 1 equiv.) of methyl iodide and 2.9 g (9 mmol, 0.04 equiv.) of TBAB. The mixture was stirred at room temperature for 30 minutes, subsequently cooled in an ice bath. Potassium tert-butoxide (25.5 g, 226.8 mmol, 1 equiv.) was added slowly. The mixture was stirred without further cooling and the free moving slurry solidified in 15-20 minutes. The mixture was suspended in 100 ml of water, and extracted with 2×100 ml of DCM. The organic phases (turbid emulsions) were filtered through impregnated phase separatory paper and evaporated. The crude product was purified by rectification, and 7.7 g (GC: 90%) of the expected product was isolated. 8 ml of hexane was added to the material. The resulting emulsion was stirred intensively, then the material crystallized. It was filtered, washed with 2×3 ml of cold hexane and dried. Yield: 4.2 g (52.4 mmol, 11.5%). GC: 95.6%.

### Synthesis of 2-{5-[(tert-Butyldimethylsilyl)oxy]pent-2-yn-1-yl}-2-methylpropanedinitrile (UB-12576)

To 2.7 g (33.6 mmol, 1 equiv.) of UB-12569 dissolved in 15 ml THF, 18.50 ml (36.96 mmol, 1.1 eq.) of NaHMDS (2M solution in THF) was added at 0° C., and agitated for 30 minutes. The mixture was heated to room temperature and stirred for an additional 30 minutes, then 12.470 g (33.60 mmol, 1 eq.) of UB-12526 was added in 30 ml of DMF. The reaction mixture was heated to 50° C. and stirred for 5 hours and checked by TLC. The reaction was quenched by addition of 200 ml of water, and the resulting mixture was extracted with 5×50 ml of ethyl acetate. The combined organic layers were washed with brine (2×50 ml) and filtered. The clear solution was evaporated to dryness and the residue was purified by column chromatography (32 g of silica gel, n-hexane:acetone=20:1). Yield: 9.1 g (32.9 mmol, 97.9%).

### Synthesis of 2-(5-Hydroxypent-2-yn-1-yl)-2-methylpropanedinitrile (UB-12577)

9.1 g (32.9 mmol) of UB-12576 was dissolved in a mixture of acetic acid:THF:water=9:2:1 (180 ml), the mixture was immersed in a 35° C. oil bath and stirred for four hours. The reaction was complete by TLC. THF was evaporated, and water (25 ml) added to the residue. The mixture was extracted with diethyl ether (4×50 ml). The combined ether extracts were washed with saturated NaHCO₃ solution (5×30 ml) and brine (2×30 ml), then evaporated to dryness. The crude product was purified by column chromatography (250 g of silica gel, DCM:methanol=10:1). Yield: 3.6 g (22.2 mmol, 67.5%). GC: 94%.

### Synthesis of 2-(5-fluoropent-2-yn-1-yl)-2-methylpropanedinitrile (UB-12626)

UB-12577 (2.50 g, 15.5 mmol, 1 equiv.) was dissolved in 20 ml of DCM and the solution was cooled to −30° C. A solution of 5 g (31.0 mmol, 2 equiv.) of DAST in DCM (10 ml) was added dropwise over 60 minutes. The reaction mixture was stirred for an additional hour at −30° C., then allowed to warm to room temperature and stirred for four hours. The reaction mixture was poured into 50 ml of ice-cooled NaHCO₃ solution and the pH of the resulting mixture was adjusted to 8 by addition of saturated Na₂CO₃ solution. The organic layer was separated and the aqueous phase extracted with DCM (3×20 ml). The combined organic phases were washed with brine, dried over Na₂SO₄ and evaporated. The crude product was purified by flash chromatography. Yield: 485 mg (2.95 mmol, 19.0%).

### Synthesis of 2-(5-Fluoropent-2-en-1-yl)-2-methylpropanedinitrile (UB-12741)

106 mg (0.64 mmol) of UB-12626 was hydrogenated in 10 ml of ethyl acetate in the presence of 4 mg of Lindlar catalyst and 40 μl of quinoline for 24 hours. The reaction was not complete by GC, so an additional 8 mg of catalyst was added and the mixture hydrogenated for 24 hours. The reaction was complete by GC, the catalyst was filtered off, and the filtrate evaporated, redissolved in 2 ml of DCM, washed with 1N hydrochloric acid, dried over Na₂SO₄ and evaporated. Yield: 60 mg (0.36 mmol, 56.3%).

### Synthesis of 5-[7-Fluoro-2-(1H-1,2,3,4-tetrazol-5-yl)hept-4-en-2-yl]-1H-1,2,3,4-tetrazole (UB-12778)

UB-12741 (45 mg, 0.27 mmol, 1 equiv.) was dissolved in 0.7 ml of N,N-dimethyl acetamide, and to the solution 76 mg (1.16 mmol, 4.3 equiv.) of sodium azide and 248 mg (1.08 mmol, 4 equiv.) of zinc bromide were added. The suspension was heated to 100° C. and stirred for 4.5 hours. The mixture was cooled, diluted with water and 1 ml of 2N HCl solution was added. The mixture was extracted with 4×1 ml of ethyl acetate, the organics were combined and evaporated. The material was dried by repeated evaporation form 5 ml of ethanol. Yield: 114 mg of crude product. The material was identified by LC-MS ([M−H]: 251).

### Synthesis of [3H]APO-600

UB-12778 (17 mg) was dissolved in 3 ml of ethanol and 3 mg of 10% Pd/C catalyst was added. The mixture was reacted with tritium gas for three hours. The catalyst was filtered off, and the mixture was evaporated. To the crude material ethanol was added and evaporated again to eliminate mobile tritium. The residue was redissolved in ethanol and purified by HPLC. The collected product fractions of five preparative injections were carefully evaporated, redissolved in 10 ml (measured by weight) of HPLC-grade ethanol and the total activity was measured and found to be 10.5 mCi (1.05 mCi/ml).

Replacement of tritium gas by hydrogen gas will lead to cold analog, APO-600.

### Example 7

### In-Vitro Screening

For in-vitro screening of the compounds of the invention, apoptosis-induction model in hematopoietic derived cancer cell line, Jurkat was used. The model is highly characterized and was shown to induce 50-60% apoptosis upon treatment.

Jurkat cells were cultured in RPMI supplemented with 10% fetal bovine serum, 4 mM L-glutamine, 100 U/ml penicillin and 100 n/ml streptomycin in a humidified incubator at 37° C., 5% CO₂. Cells were counted and re-suspended in hepes buffer saline (HBS).

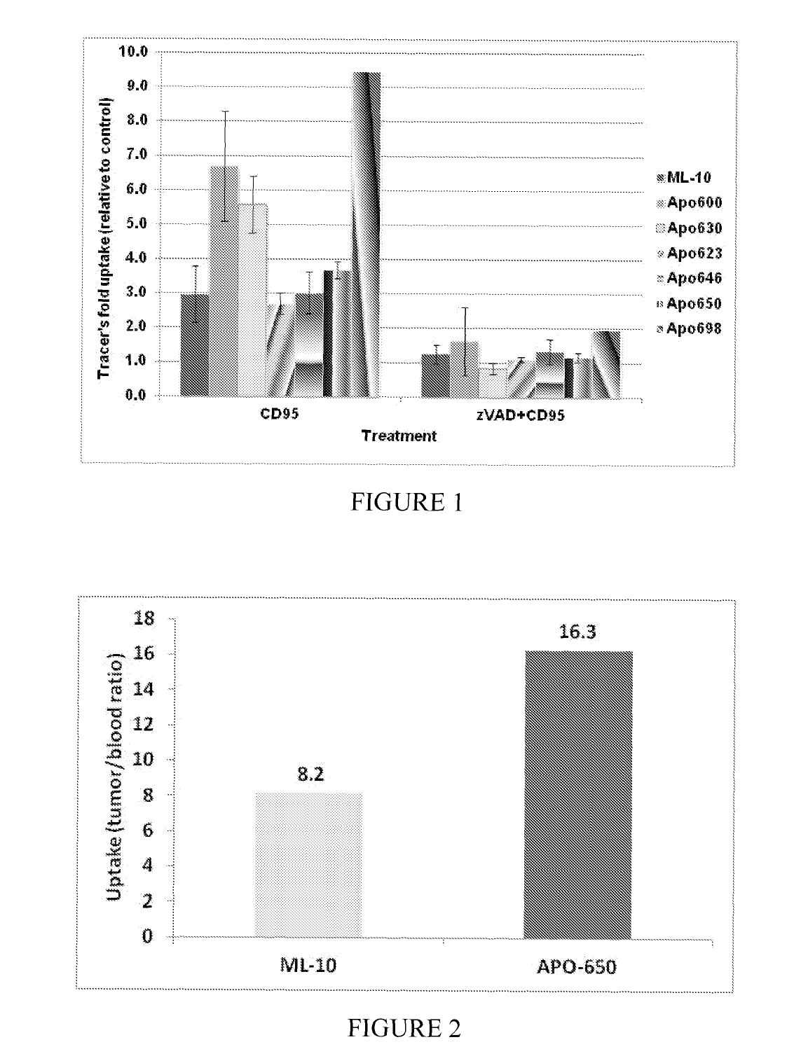

Apoptosis induction: 10⁷ cells/ml were incubated for two hours with 0.1 n/ml of anti-Fas (CD95) antibody. For apoptosis inhibition (specific control), induced and control cells were incubated with 50 μM of the specific caspase inhibitor, Zvad. 2 μl Ci of the tritiated compounds or ML-10 as reference, were added to the cells for 30 minutes. All incubations were performed at 37° C. After incubation with the tritiated compounds, cells were washed twice in HBS, transferred to scintillation liquid and radioactivity was monitored using beta-counter. The percentage of uptake was calculated using the standard DPM readings. Control untreated uptake value was set as 1 and the fold increase uptake of CD95-treated cells was used to determine specific accumulation into apoptotic cells. As can be seen in FIG. 1, three candidates (APO-600, APO-630 and APO-698) demonstrated a high uptake of the tracer into the apoptotic cells. The uptake was 2-3 higher compared to ML-10 (FIG. 1).

### Example 8

### In-Vivo Screening

In order to assess the in-vivo accumulation of ML-10 and compounds of the invention, the uptake into human colorectal cancer cells HCT-116 xenografts was monitored. Athymic nude mice were subcutaneously inoculated with 106 HCT-116 cells. After the formation of tumors, ML-10 and APO-650 were intravenously administrated to the mice, 30 and 90 minutes before scarification (n≧8). Peripheral blood samples were collected prior to animals' scarification. Tumors were dissected, homogenized and all samples were calibrated in scintillation fluid before radioactivity was measured in a beta-counter. To inspect signal to noise ratio, the ratio of uptake into the tumor versus blood was calculated. As can be seen from FIG. 2, APO-650 demonstrated a six-fold tumor/blood ratio indicating an increased retention in target cells and/or rapid clearance from the blood.

### Example 9

### In-Vivo Screening

In order to determine the pharmacokinetics properties of APO-698, its unlabeled analog, APO-697 was used. Table-1 summarizes the pharmacokinetic study performed in CD1 mice for the two unlabeled (cold) analogs, ML-10 and APO-697. As can be seen, APO-697 demonstrated shorter t_{1/2} as well as higher volume of distribution and total clearance compared to ML-10. Blood partitioning values showed low partitioning to red blood cells of both compounds (Table-2). Pharmacokinetic evaluation of two tritiated candidates revealed a shorter half life for both APO-630 and APO-650 in chemotherapy treated mice and for APO-650 in naive mice (Table-3).

TABLE 1
Pharmacokinetics of ML-10 and APO-697

| | ML-10 | APO-697 |
|---|---|---|
| t_{1/2} (min) | 11.16 | 7.92 |
| Total clearance (L/hr/kg) | 0.613 | 3.88 |
| | (11.4%) | (71.9%) |
| Vss (L/kg) | 0.152 | 0.435 |
| | (21.0%) | (60.0%) |
| AUCINF (hr*ng/mL) | 32.6 | 5.16 |

TABLE 2
Blood partitioning of MC-10 and APO-697

| Test Article | Species | KRBC/PL |
|---|---|---|
| ML-10 | mouse | 0.01 |
| APO-697 | mouse | 0.07 |

TABLE 3
Pharmacokinetic evaluation of APO-650 and APO-630

| | t_{1/2} (min) | | |
|---|---|---|---|
| | ML-10 | APO-650 | APO-630 |
| Naive | 10 | 8 | 10 |
| CPT-11 treated | 10 | 5 | 6 |

1-60. (canceled)

61. A compound represented by formula (V): 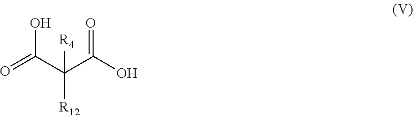
wherein R₄ is selected from C₁, C₂, C₃, C₄, C₅, C₆, C₇, C₈ alkyl, and R₁₂ is selected from a C₁, C₂, C₃, C₄, C₅, C₆, C₇, C₈ C₉ alkyl or alkylene attached to a tubulin ligand, wherein either R₄ or R₁₂ is further optionally attached to a leaving group, a leaving group attached to a C₁, C₂, C₃, C₄, C₅ or C₆-alkyl, a leaving group attached to a C₁, C₂, C₃, C₄, C₅ or C₆-alkoxy, a marker for diagnostics or a therapeutic agent.

62. The compound according to claim 61, wherein the leaving group is selected from halide, sulfonate, mesylate, tosylate, triflate, nosylate, brosylate or a phenyl substituted by a nitro or halogen.

63. The compound according to claim 61, which is further radiolabeled.

64. The compound according to claim 63, for use in X-ray, CT scan, magnetic resonance imaging (MRI), single photon emission tomography (SPECT) and positron emission tomography (PET).

65. The compound according to claim 61, radiolabeled with ¹⁸F.

66. The compound according to claim 61, represented by formula Vfp 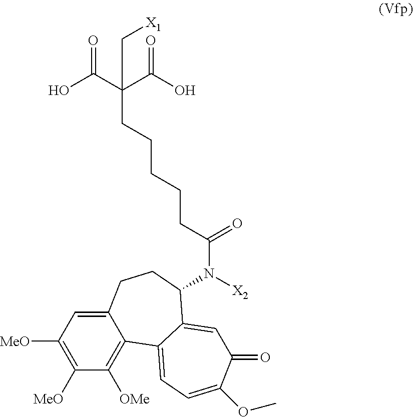
wherein each of X₁ and X₂ is independently selected from H, a leaving group, a leaving group attached to a C₁, C₂, C₃, C₄, C₅ or C₆-alkyl, or a leaving group attached to a C₁, C₂, C₃, C₄, C₅ or C₆-alkoxy; provided that both X₁ and X₂ are not simultaneously H, wherein the compound is optionally ¹⁸F radiolabeled by a ¹⁸F or ¹⁸F—C₁, C₂, C₃, C₄, C₅, or C₆-alkyl.

67. The compound according to claim 61, represented by formula Vf′ or Vf″.

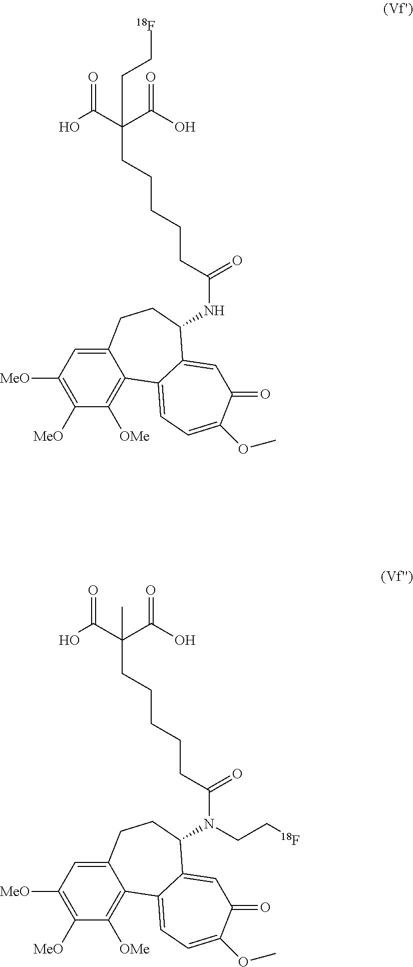 [SMILES: COc1cc2c(c(OC)c1OC)-c1ccc(OC)c(=O)cc1[C@@H](NC(=O)CCCCCC(CC[18F])(C(=O)O)C(=O)O)CC2 ; COc1cc2c(c(OC)c1OC)-c1ccc(OC)c(=O)cc1[C@@H](N(CC[18F])C(=O)CCCCCC(C)(C(=O)O)C(=O)O)CC2]

68. A compound represented by the structure in formula (VIII):
R—O—X (VIII)
wherein R stands for any of the compounds according to claim 61; and
X is a leaving group.

69. The compound according to claim 68, wherein X is a halide or a sulfonate.

70. The compound according to claim 69 represented by formula VIII″ 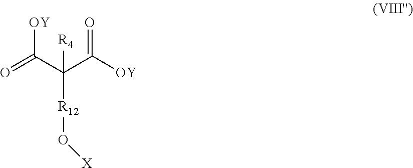
wherein R₄ is H or a C₁, C₂ or C₃ alkyl and R₁₂ is a linear or branched C₁, C₂, C₃, C₄, C₅, C₆, C₇, C₈, or C₉-alkyl or alkylene group, optionally substituted with an aryl or heteroaryl comprising one or two rings; Y is a protecting group selected from the group consisting of methyl, ethyl, tert-butyl, benzyl and X is a leaving group.

71. The compound according to 70, wherein X is a halogen or a sulfonate.

72. A method for the selective detection of cells undergoing perturbations and alterations of their normal plasma membrane organization among a population of cells, wherein the method comprises contacting the cell population with a compound according claim 61, said method comprising the detection of the signal obtained from said cells by a suitable detector.

73. A method for selective detection of cells undergoing cell death in the living body of a multi-cellular organism, said method comprising administration to said organism adequate amounts of a compound according claim 61, wherein said compound is radiolabeled, said method comprising measuring the signal obtained from said marker by a suitable method for detection.

74. The method according to claim 73, for the detection of cells undergoing apoptosis.

75. The method according to claim 73, for use for an indication selected from detection of foci of disease, characterizing disease severity, or monitoring of response to treatment; through molecular imaging of the related cell death, wherein the disease is selected from cancer, degenerative disease, inflammatory disease, immune-mediated disease, or a toxic insult.
